(19) United States
(12) Patent Application Publication
(10) Pub. No.: US 2024/0392318 A1
(43) Pub. Date: Nov. 28, 2024

(54) HOST CELL LINES AND METHODS FOR IDENTIFYING AND USING SUCH HOST CELL LINES

(71) Applicant: SANOFI, Paris (FR)

(72) Inventors: Barak Balva, Cambridge, MA (US); Christine DeMaria, Cambridge, MA (US); Karen Vincent, Cambridge, MA (US); Saurabh Sen, Cambridge, MA (US); Patricia Alves, Oeiras (PT); Mafalda Dias, Oeiras (PT); Jose Escandell, Oeiras (PT); Daniel Pais, Oeiras (PT)

(21) Appl. No.: 18/647,572

(22) Filed: Apr. 26, 2024

Related U.S. Application Data
(60) Provisional application No. 63/462,215, filed on Apr. 26, 2023.

(30) Foreign Application Priority Data
Apr. 4, 2024 (EP) 24315118.0

(51) Int. Cl. C12N 15/864; C12N 5/09; C12N 7/00; C12N 15/861

(52) U.S. Cl. CPC C12N 15/8645; C12N 5/0693; C12N 7/00; C12N 15/861; C12N 2513/00; C12N 2750/14143

(57) ABSTRACT

Disclosed are host cell lines and producer cell lines derived from a HeLaS3 parental cell lines and methods of producing the host cell lines and producer cell lines.

### CROSS-REFERENCE TO RELATED APPLICATIONS

This application claims priority to European Patent Application No. 24315118.0, filed Apr. 4, 2024, and U.S. Provisional Patent Application Ser. No. 63/462,215, filed Apr. 26, 2023, the disclosures of which are hereby incorporated by reference in their entirety.

### FIELD OF THE APPLICATION

The disclosure relates to methods and cell lines for producing adeno-associated virus (AAV) particles. Also disclosed are host cells derived from HeLa cell lines.

### BACKGROUND

Mammalian cell lines, such as CHO cells, HEK293, Human cervical carcinoma (HeLa) cell lines are commonly used in the biotechnology industry to produce biological molecules, such recombinant AAV (rAAV) vectors, and in some cases proteins. HeLa cells (e.g., HeLaS3) that are adapted for growth in suspension in a serum free medium are desirable for the development of large-scale cell culture processes for the manufacture of rAAV for, e.g., use in human gene therapy.

There continues to be a need to develop host cells that exhibit suitable attributes for the efficient production of rAAV including host cells that are developed without the use of animal derived products (e.g. serum) to reduce the risk of contamination.

### SUMMARY

The disclosure provides, at least in part, compositions and methods of using HeLa cells.

In one aspect, the disclosure provides a host cell line derived from a HeLaS3 parental cell line. The host cell line can have about a 0.5% to about a 25% difference in cell doubling time (hours) compared to the parental cell line. The host cell line can have about a 0.5% to about a 25% difference in transfection efficiency compared to the parental cell line. The host cell line can have about a 0.5% to about a 25% difference in peak viable cell density compared to the parental cell line. The host cell line can have about a 0.5% to about a 10% difference in percent cell viability after one cell freeze thaw cycle compared to the parental cell line. The host cell line can be transfected with one or more nucleic acid molecules encoding a heterologous transgene flanked by AAV ITRs, an AAV rep, and an AAV cap and can have about a 1- to 20-fold difference in Adeno-associated virus (AAV) vector production titer (vg/mL) compared to the parental cell line.

In one aspect, a host cell line can be selected by a method that includes: (a) expanding one or more population(s) of the HeLaS3 parental cell line in a serum-free media; (b) selecting and isolating one or more single cell clones from step (a); (c) expanding each of the one or more single cell clones from step (b) in a serum-free media; (d) selecting from each of the one or more single cell clones from step (c) and analyzing the one or more single cell clones for at least one of the following characteristics: (i) cell doubling time, (ii) transfection efficiency, (iii) peak viable cell density; and (iv) any combination thereof; and (e) growing the one or more single cell clones selected from step (d) in a serum-free media; thereby resulting in the host cell line. The one or more single cell clones can be further analyzed for the characteristics of cell outgrowth after seeding at low cell density, rAAV production titers, or combinations thereof.

In one aspect of the method, step (d) further comprises evaluation of recovery from a cell freeze-thaw cycle, a degree of cell clumping, or analysis of a metabolic profile. The metabolic profile can include measuring depletion of glucose or glutamine or secretion of lactate or any combination thereof into the serum-free media for 1-7 days. In one aspect of the method, the one or more single cell clones from step (d) can have about a 0.5% to about a 25% difference in cell doubling time (hours) compared to the parental cell line; have about a 0.5% to about a 25% difference in peak viable cell density compared to the parental cell line; and/or have about a 0.5% to about a 25% difference in percent cell viability after one cell freeze thaw cycle compared to the parental cell line. In one aspect of the method, the one or more single cell clones from step (d) can have about a 1- to 20-fold difference in AAV vector production titer (vg/mL) when transfected with one or more nucleic acid molecules encoding a heterologous transgene flanked by AAV ITRs, an AAV rep, and an AAV cap, and infected with a helper virus, as compared to the parental cell line. In one aspect of the method, the one or more single cell clones from step (c) can have about a 0.5% to about a 25% difference in transfection efficiency compared to the parental cell line. In one aspect of the method, step (d) further comprises a selection based on a principal component analysis. In one aspect of the method, the host cell line is grown in suspension.

In one aspect a recombinant AAV (rAAV) particle is produced by a method comprising: (a) transfecting a host cell as described herein under conditions to generate the rAAV particles, wherein the host cell line is transfected with one or more nucleic acid molecules encoding a heterologous transgene flanked by inverted terminal repeats (ITRs), an AAV rep, an AAV cap, and optionally a selectable marker; and expanding the one or more population(s) from the transfected parental cell line; (b) providing or infecting the cells with a AAV helper virus or a derivative thereof; and (c) harvesting the rAAV particles. The rAAV particle host cell line can be transfected with a nucleic acid molecule encoding AAV cap, which is derived from AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV V708K, a goat AAV, AAV1/AAV2 chimeric, bovine AAV, or mouse AAV capsid rAAV2/HBoV1 or variants thereof. The AAV helper virus can be Ad5 such as human Ad5. The host cell line can be infected with a helper virus. The host cell line can be infected with a helper virus that includes adenovirus, herpes simplex virus, vaccinia virus, or cytomegalovirus. The transfected host cell line can be harvested to collect AAV particles. The one or more nucleic acid molecules can be stably transfected into the host cell line.

In one aspect is a method for generating a producer cell line candidate for production of recombinant AAV (rAAV) particles. The method can comprise: (a) stably transfecting the host cell line in serum free medium with one or more nucleic acids encoding (i) a heterologous transgene flanked by two AAV inverted terminal repeats, (ii) an AAV rep gene and an AAV cap gene to generate a producer cell line, (b) infecting the producer cell line with an AAV helper virus to produce rAAV particles; and (c) selecting the producer cell line as a candidate for production of rAAV particles if the producer cell line generates a titer of rAAV particles of at least about 1E9 vg/mL. The method can further comprise a step of of expanding the producer cell line of step (c) to a cell density at or higher than 3.5E5 cells; and (d) selecting the producer cell line as a candidate for production of rAAV particles if the producer cell line generates a titer of rAAV particles of at least about 1E9 vg/mL following step (d). The method can further comprise a step of expanding the cells to a cell density at or higher than 3.5E5 cells and selecting the producer cell line as a candidate for production of rAAV particles if the producer cell line generates a titer of rAAV particles of at least about 1E10 vg/mL. The method can further include determining the rAAV titer by quantitative polymerase chain reaction (qPCR). The method can further determine the cell viability or cell viability density by freeze-thawing, shear stress, or combination thereof. The method of determining cell viability can comprise selecting cells that have a cell viability greater than or equal to 70% cell viability compared to the HeLaS3 parental cell line. Cells can be selected having a viability greater than or equal to 90% cell viability as compared to the HeLaS3 parental cell line. Cells can be selected that have a cell doubling time less than or equal to 32 hours viability as compared to the HeLaS3 parental cell line. Cells can be selected that have a cell doubling time less than or equal to 32 hours viability compared to the HeLaS3 parental cell line. Cells can be selected that have a peak viable cell density greater than or equal to 3×10⁶ cell/mL viability compared to the HeLaS3 parental cell line. Cells can be selected that have greater than or equal to 30 percent of cells transfected compared to the HeLaS3 parental cell line. Cells can be selected that have reduced clumping of cells visible to the naked eye compared to the HeLaS3 parental cell line. The producer cell line can be a mammalian host cell. The producer cell line can be a HeLa3 cell line. The serum-free medium used to generate the cells can be free of any animal derived components. The serum-free medium can include medium supplemented with glutamine. The serum-free medium can include medium supplemented with about 6 mM of glutamine.

In one aspect is a method of producing a host cell line comprising the steps of: (a) expanding one or more population(s) of a HeLaS3 parental cell line at a density of 0.5 cells/well in media comprising EX-CELL HeLa growth medium supplemented with 6 mM L-Glutamine, 50% DMEM/F-12 (supplemented with 6 mM L-Glutamine), 20% conditioned media and 1× InstiGRO CHO supplement, wherein all components of the media and conditioned media are serum free and free of animal derived components; (b) selecting and isolating one or more single cell clones from step (a); (c) expanding each of the one or more single cell clones from step (b) in a serum-free media; (d) selecting from each of the one or more single cell clones from step (c) for at least one of the following characteristics: (i) a cell viability, (ii) a cell doubling time, (iii) a transfection efficiency, (iv) a peak viable cell density; (v) clumping; (vi) any combination thereof and (e) isolating and expanding a single cell selected from step (d) in a serum-free media thereby producing the host cell line. The method can comprise further selection for cell outgrowth after seeding at low cell density; rAAV production titers; and combinations thereof.

In one aspect provides a method of generating a producer cell line comprising the steps of (a) transfecting any of the host cell lines described herein or a host cell line that has been selected by any of the methods described herein with one or more nucleic acids encoding (i) a heterologous transgene flanked by two AAV inverted terminal repeats, (ii) an AAV rep gene and an AAV cap gene, (iii) and AAV helper genes (or infecting the host cell with helper virus); and (b) selecting the producer cell line where the producer cell line generates a titer of rAAV particles of at least about 1E9 to about 1E11 vg/mL.

One aspect provides a method for making recombinant adeno-associated virus (rAAV), comprising the steps of: (a) stably transfecting any of the host cell lines described herein or a host cell line produced by any of the methods described herein with one or more nucleic acids encoding (i) a heterologous transgene, (ii) an AAV rep gene and an AAV cap gene, and (iii) AAV inverted terminal repeats (ITR); (b) infecting the host cell with a helper virus; and (c) isolating the rAAV particles wherein the titer of the rAAV particles produced is at least about 1E9 vg/mL The titer of the rAAV particles can be from about 1E9 vg/mL to about 1E11 vg/mL.

### BRIEF DESCRIPTION OF THE FIGURES

The foregoing and other features and advantages of the disclosure will be more fully understood from the following detailed description of illustrative aspects taken in conjunction with the accompanying drawings.

FIG. 1 shows a flow diagram for isolation and initial selection of HeLaS3 derived host cells and depicts the workflow for isolating and selecting host cell lines with desirable attributes. First, the HeLaS3 parental line was subcloned into 96-well plates to achieve a seeding density of ≤1 cell per well. For wells with clonally derived cell growth, the cells were expanded to 24-well plates, then to 6-well plates, then to shake flasks. The medium used was animal derived component free (viz., no animal derived components or products). Each subclone was cryopreserved into multiple cryovials. Subclones were thawed and screened for freeze-thaw robustness (including low degree of clumping) and cell growth properties, resulting in 54 lead candidate subclones for further evaluation.

FIG. 2 is a schematic illustrating some of the favorable cell attributes presented by the improved host cell lines embodied by the present disclosure. These factors include improved growth attributes such as population doubling time (PDT) and batch culture peak viable cell density (VCD), increased transfection efficiency with large plasmids of approximately 4, 5, 10 or more kilobases, and favorable viral vector titer production.

FIG. 3 outlines the selection of improved host cell lines selected from 54 candidate cell populations. Initially, 54 candidate cell populations were isolated from HeLa parental cells as shown in FIG. 1. These populations were then narrowed to 23 candidate cell populations based on a screening procedure which evaluated parameters such as freeze thaw tolerance, population doubling time, transfection efficiency, degree of cell clumping, and peak VCD. The 23 candidate cell populations were then grown in serum-free medium and further subjected to additional screening encompassed by culturing the 23 candidate cell populations in an automated bioreactor system (labeled “process fit evaluation (Ambr®)” plus a Shear stress test) which allowed for a further narrowing of 5 candidate cell populations.

FIG. 4A-B depict the candidate cell populations evaluated in a freeze-thaw tolerance (FTT) assessment. FIG. 4A is a schematic of the freeze thaw tolerance (FTT) assay. Thirty frozen candidate cell populations were thawed on day 0. The candidate cell populations were grown in liquid culture and passaged twice (passage 1 and 2 are labeled, “P1 FTT1” and “P2 FTT2,” respectively). Two replicates from FTT1 and FTT2 were tested for cell viability. FIG. 4B is a graph measuring the cell percent viability from the FFT1 and FFT2 samples. The parental HeLa cell line was used as a control (dashed box labeled, “HeLa parental”).

FIG. 5A-B depict the candidate cell populations evaluated in a population doubling time (PDT) assessment. FIG. 5A is a schematic of the population doubling time (PDT) assay. Using the same experimental set up as the FTT assay, the rate at which candidate populations doubled was measured in hours. FIG. 5B is a graph measuring the rate at which the candidate cell population doubled over two passages. The parental HeLa cell line was used as a control (dashed box labeled, “HeLa parental”).

FIG. 6A-B depict the candidate cell populations evaluated in a nucleofection transfection efficiency assessment. FIG. 6A is a schematic of the nucleofection transfection (NTx) efficiency assay. The candidate populations were transiently transfected with plasmids carrying the fluorescent reporter, GFP. FIG. 6B is a graph measuring the percent of GFP positive cells calculated 24-hours post transfection. The parental HeLa cell line was used as a control (dashed box labeled, “HeLa parental”).

FIG. 7A-B depict the candidate cell populations evaluated in a peak viable cell density (VCD) assessment. FIG. 7A is a schematic of the peak viable cell density (VCD) assay where candidate populations were thawed and grown over two passages (P1—replicate 1 and P2—replicate 2). FIG. 7B is a graph calculating the normalized fold change (relative to the HeLa parental) in viable cell density for the 32 candidate populations. The parental HeLa cell line was used as a control (dashed box labeled, “HeLa parental”).

FIG. 8A-B depict a principal component analysis (PCA) plot obtained for candidate cell populations after the first relative clone attributes screen. FIG. 8A is an exemplary PCA biplot for all thirty-two candidate populations (represented by individual dots). FIG. 8B is a graph of and exemplary score contribution weights for the candidate cell populations for the FTT, PDT, and peak VCD. The GFP transfection efficiency was assessed for 24 hours and 48 hours. PCA is a multivariate tool that combines all the original variables and identifies correlations (structured variation) leaving out the noise (unstructured variation).

FIG. 9 depicts a timeline when the medium and cell candidate populations are added to a small scale bioreactor (such as an Ambr® 15) for cell growth kinetics and metabolic profiling analysis.

FIG. 10A-B report the cell growth assessment of 21 candidate populations cultured over 7 days under small-scale bioreactor conditions (such as an Ambr® 15 bioreactor). FIG. 10A is a line graph measuring the viable cell density (E6 cells/ml) for each candidate cell population and FIG. 10B is a line graph measuring the percent viability for each candidate cell population over 7 days.

FIG. 11A-B report the cell metabolic profiling of 21 candidate populations cultured over 7 days under small-scale bioreactor conditions. FIG. 11A is a line graph measuring the rate of glucose consumption for each candidate cell population and FIG. 11B is a line graph measuring the rate of lactate production by each candidate cell population over 7 days.

FIG. 12 reports the cell growth assessment measured in terms of peak VCD and cell viability and the metabolic profiling measured in terms glutamine depletion, glucose consumption, and lactate production from the top 7 out of 21 candidate cell populations cultured over 7 days under small-scale bioreactor conditions.

FIG. 13A-13B measure the amount of AAV titer (vg/mL) released into the cell culture medium from each of the top 7 candidate cell populations 72 hours post media change, which was day 5 post transfection with a viral plasmid. FIG. 13A is a schematic of an assay in which at Day 1 a candidate population is transfected with a viral plasmid, at day 2 (24 h post transfection) the culture medium is changed, and at day 5 (72 h harvest) the cell culture medium is harvested for viral titer analysis. FIG. 13B is a graph of the amount of AAV titer (vg/mL) released into the cell culture medium from each of the top 7 candidate cell populations at day 5 with the viral plasmid.

FIG. 14 depicts one aspect of the process to generate AAV producer cell lines starting from a HeLaS3 host cell line. First, a HeLaS3 host cell is transfected with a heterologous transgene, a AAV rep, a AAV cap, a selectable marker using a plasmid (see diagram labels, “plasmid components” and “transfection”). Transfected cells are cultured in the presence of a selection agent, and stably transfected cell populations are expanded, cryopreserved, and screened for productivity. The lead populations (based on several metrics that include growth and productivity) are seeded into well-plates to isolate single cell clones, which are then expanded, cryopreserved, and screened for desired attributes.

FIG. 15 depicts an exemplary clone characterization process workflow.

FIG. 16 graphically depicts the transfection efficiency represented as the percentage of green fluorescent protein (GFP) cells (solid black bars) and cell viability (white bars) of subclones and HeLaS3 parental cells.

FIG. 17 depicts the process for evaluating cell growth performance in the cell culture bioreactor system, Ambr® 15.

FIG. 18 depicts the process for evaluating cell growth performance and viability in the cell culture bioreactor system, Ambr® 15 under shear stress test.

FIG. 19 graphically depicts the viable cell density and cell viability profiles of 11 top clones evaluated for shear stress tolerance. VCD (solid black full lines) and cell viability (dashed lines) profiles of HeLaS3 parental cells were included as control. Increasing shear rates and % of DO (black circles) were applied to increase the shear stress of cell cultures. Clones M. Q, E, S, and P presented high tolerance to shear stress and, in combination with other attributes were classified as the five top clones.

FIG. 20A-B depicts graphically the top five selected clones for nucleofector efficiency or viability and CAR levels.

FIG. 21 depicts graphically the viable cell density (VCD) and cell viability (Viab) from Ambr®15 runs performed for clone M and the parental HeLa line. Shear stress was induced by increasing the stirring rate and dissolved oxygen (DO) set point. The shear stress run is depicted as the dark black line. The average of 3 runs in Ambr® 15 for VCD and viability are shown with the HeLa parental line used as a control.

FIG. 22 depicts graphically viable cell density (VCD) and cell viability (Viab) from Ambr®15 runs performed for clone R and the parental HeLa line. Shear stress was induced by increasing the stirring rate and dissolved oxygen (DO) set point. The shear stress run is depicted as the dark black line. The average of 3 runs in Ambr®15 for VCD and viability are shown with the HeLa parental line used as a control.

FIG. 23 depicts graphically the viable cell density (VCD) and cell viability (Viab) from Ambr®15 runs performed for clone Q and the parental HeLa line. Shear stress was induced by increasing the stirring rate and dissolved oxygen (DO) set point. The shear stress run is depicted as the dark black line. The average of 3 runs in Ambr®™15 for VCD and viability are shown with the HeLa parental line used as a control.

FIG. 24 depicts graphically the viable cell density (VCD) and cell viability (Viab) from Ambr®15 runs performed for clone C and the parental HeLa line. Shear stress was induced by increasing the stirring rate and dissolved oxygen (DO) set point. The shear stress run is depicted as the dark black line. The average of 3 runs in Ambr®15 for VCD and viability are shown with the HeLa parental line used as a control.

FIG. 25 depicts graphically the viable cell density (VCD) and cell viability (Viab) from Ambr®15 runs performed for clone E and the parental HeLa line. Shear stress was induced by increasing the stirring rate and dissolved oxygen (DO) set point. The shear stress run is depicted as the dark black line. The average of 3 runs in Ambr®15 for VCD and viability are shown with the HeLa parental line used as a control.

FIG. 26 depicts graphically the plating efficiency of selected clones in serum free conditions. HeLaS3 parental cells and Clones M, B, Q, E, and C were seeded at low cell density (3 cells/well) using a cell dispenser equipment. Their capability of cell colony outgrowth was evaluated after 14 days of deposition. The graph represents the percentage of colony outgrowth, calculated as the number of counted colonies divided by the number of cells seeded, multiplied by 100.

### DETAILED DESCRIPTION

Disclosed are host cell lines and producer cell lines derived from a HeLaS3 parental cell line. The host cell lines are derived from the HeLaS3 parental cell and have attributes that differ from the parental cell line. In some aspects host cell lines have improved characteristics such as in doubling time, improved transfection efficiency, peak viable cell density, percent cell viability after one cell free thaw cycle, AAV vector production titer (vg/mL), reduced clumping, improved sheer stress, or combinations thereof when compared to the HeLaS3 parental cell line. In some aspects, the host cell is a cell derived from a single cell.

### General Techniques

Certain techniques and procedures described or referenced herein are described in Molecular Cloning: A Laboratory Manual (Sambrook et al., 4^{th} ed., Cold Spring Harbor Laboratory Press, ColdSpring Harbor, N.Y., 2012); Current Protocols in Molecular Biology (F. M. Ausubel et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002);

Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic
Publishers, 1995); and DeVita Jr, V. T., Lawrence, T., & Rosenberg, S. A. (2012). Cancer: principles & practice of oncology: annual advances in oncology. Lippincott Williams & Wilkins.

### Definitions

Unless defined otherwise, all technical and scientific terms used herein have the same meaning as commonly understood by one of ordinary skill in the art to which this disclosure belongs.

A “vector,” as used herein, refers to a recombinant plasmid or virus that comprises a nucleic acid molecule to be delivered into a host cell, either in vitro or in vivo.

The term “polynucleotide” or “nucleic acid molecule” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically, or biochemically modified, non-natural, or derivatized nucleotide bases. The backbone of the polynucleotide can comprise sugars and phosphate groups (as may typically be found in RNA or DNA) or modified or substituted sugar or phosphate groups.

The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

A “recombinant viral vector” refers to a polynucleotide viral vector comprising one or more heterologous nucleic acid molecules (i.e., nucleic acid molecules not naturally associated with the viral vector). In the case of recombinant AAV viral vectors, a heterologous nucleic acid molecule can be flanked by two ITRs.

A “recombinant AAV vector (rAAV vector)” refers to a polynucleotide vector comprising one or more heterologous nucleic acid molecules (i.e., nucleic acid molecules not naturally associated with the wild-type AAV vector) that are flanked by two AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a rAAV vector is incorporated into a larger polynucleotide (e.g., in a chromosome or in another vector such as a plasmid used for cloning or transfection), then the rAAV vector can be referred to as a “pro-vector” which can be “rescued” by replication and encapsidation in the presence of AAV packaging functions and suitable helper functions. A rAAV vector can be in any of several forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

A “host cell line” refers to a cell population capable of continuous or prolonged growth and division in vitro. Host cell lines can have spontaneous or induced changes that can occur in karyotype during storage or transfer of the cell lines. Therefore, host cell lines may not be identical to the ancestral cells or cultures and therefore the host cell line can include variants.

A “producer cell line” refers to cells derived from a host cell line. Producer cells can be generated by integrating (e.g., stably integrating) one or more AAV genes (e.g., rep and cap) along with an ITR flanked transgene of interest into a host cell.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it can be transcribed into a molecule that mediates RNA interference, such as miRNA, siRNA, or shRNA.

An “AAV inverted terminal repeat (ITR)” sequence refers to relatively short sequences (e.g. approximately 145-nucleotide sequence) found at the termini of viral genomes which are in opposite orientation. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity (designated A, A′, B, B′, C, and C′ regions), allowing intrastrand base-pairing to occur within this portion of the ITR.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. Several such helper viruses have been identified, including adenoviruses, herpesviruses, poxviruses such as vaccinia and baculovirus. The adenoviruses encompass several different subgroups, although Adenovirus type 5 of subgroup C (Ad5) is most used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions. Baculoviruses available from depositories include Autographa californica nuclear polyhedrosis virus.

### Host Cells

In some aspects, a host cell line or producer cell line is derived from a HeLaS3 parental cell line. In some aspects, a HeLaS3 cell line is a subclone of a HeLa cell line. In some aspects, a HeLaS'parental line is a clonal derivative of the parental HeLa cell line (ATCC CCL-2). In some aspects, a host cell is derived from a HeLaS3 parental cell line (e.g. derived from a single cell or single progenitor). In some aspects, a host cell or producer cell derived from a HeLaS3 parental cell line can be manipulated to develop novel cell phenotypes that can be useful for large-scale production of recombinant AAV viral vectors.

In some aspects, a host cell or producer cell is adapted for cultivation in a suspension, without serum, at high cell densities, and at large-scale (e.g., greater than 10, 15, 20, or 50 liters of culture media or greater than least 5, 10, 15, or 20 m² of culture media.

In some aspects, suspension culture has the advantage of large scale up because such cells have the advantages of ease of manufacture, less expense, requirement for less space with no need for the use of proteolytic enzymes, and the capability of culture in bioreactors with a total environmental control. Moreover, suspension cell culture can be characterized by homogeneity of nutrients and uniform cell population with good reproducibility from experiment to experiment. In some aspects, the host cell or producer cell is grown in a serum-free medium. In some aspects, the host cell or producer cell is grown in media with no animal derived components or products. In some aspects, the host cell or producer cell is grown in media with no mammalian or avian derived components or products. The media can, in some aspects, have animal derived products that are biosafe and pose no risk of contamination of transmissible spongiform encephalopathy (TSE) or bovine spongiform encephalopathy (BSE) (e.g., cod liver oil). In some aspects, the host cell or producer cell media is free of animal derived components except for cod liver oil that is biosafe and free of TSE/BSE. In some aspects, the host cell or producer cell is grown in Ex-CELL® HeLa Serum-Free medium (Sigma-Aldrich, St. Louis, USA). In some aspects, the media (e.g. Ex-CELL® HeLa Serum-Free medium) is supplemented with glutamine at, for example 4, 5, 6, or 7 mM.

In some aspects, methods of selecting a host cell line is described in the Examples section. In some aspects, a producer cell is selected by the method of:
(a) expanding one or more population(s) of the HeLaS3 parental cell line in a serum-free media;
(b) selecting and isolating one or more single cell clones from step (a);
(c) expanding each of the one or more single cell clones from step (b) in a serum-free media;
(d) selecting from each of the one or more single cell clones from step (c) and analyzing the one or more single cell clones for at least one of the following characteristics:
   (i) cell doubling time,
   (ii) transfection efficiency, and
   (iii) peak viable cell density; and
(e) growing the one or more single cell clones selected from step (d) in a serum-free media; thereby resulting in the host cell line;
(f) transfecting the host cell line that has been selected by steps (a)-(e) with one or more nucleic acids encoding (i) a heterologous transgene flanked by two AAV inverted terminal repeats, (ii) an AAV rep gene and an AAV cap gene; and
   (g) selecting the producer cell line where the producer cell line generates a titer of rAAV particles.

In some aspects a host cell line can be transfected with one or more nucleic acid molecules encoding a heterologous transgene flanked by two AAV inverted terminal repeats, an AAV rep, an AAV cap, and optionally, a selectable marker and/or nucleic acid sequences that encode adenovirus helper virus genes to obtain a producer cell line. The AAV titer per cell can be quantified.

In some aspects, a host cell line is obtained comprising the steps of:
(a) expanding one or more population(s) of the parental cell line in a serum-free media;
(b) selecting and isolating one or more single cell clones from step (a)
(c) expanding each of the one or more single cell clones from step (b) in a serum-free media;
(d) selecting from each of the one or more single cell clones from step (c) at least one of the following characteristics:
   (i) cell viability,
   (ii) cell doubling time,
   (iii) transfection efficiency,
   (iv) peak viable cell density; and
   (v) clumping; and
(e) isolating and expanding a single cell selected from step (d) in a serum-free medium thereby generating the host cell line.

In some aspects, a host cell line is obtained by repeating steps (d)-(e) until desired to select a single cell for further isolating and expanding in a serum-free medium thereby generating the host cell line.

In some aspects, a host cell line can be alternatively or additionally selected for at least one of the characteristics: (i) a cell viability; (ii) a cell doubling time, (iii) transfection or nucleofection efficiency, (iv) peak viable cell density; (v) clumping; (vi) metabolic profiling (e.g. changes in lactate and glucose consumption); (vii) cell outgrowth after seeding at low cell density (e.g. 0.5, 1, 2, 3, 4, or 5 cells/well); (viii) rAAV production titers; (ix) rAAV product quality which, in some cases, is assessed by percent full capsids; or (x) any combination thereof. In some aspects, the method steps (d) to (e) can be repeated multiple times to obtain a desired characteristic. In a particular round of selection, the same or different characteristics or a combination of characteristics can be selected. For example, in a first selection round, the cells can be selected for cell viability. In a second selection round, the cells can be selected for cell viability and a different characteristic such as cell doubling time. In a third selection round the cells can be selected for the cell viability, doubling time and yet another characteristic such as clumping and so forth. In some embodiments, the characterizations of the parameters for selection were carried out in parallel and clones were selected based on an overall evaluation of the parameters (e.g., cell viability, doubling time, transfection or nucleofection efficiency, peak viable cell density, clumping, metabolic profiling, cell outgrowth after seeding at low cell density, or rAAV production titers).

In some aspects, a host cell line is produced by a method comprising the steps of:
(a) expanding one or more population(s) of a HeLaS3 parental cell line at a density of 0.5 cells/well in medium comprising EX-CELL HeLa growth medium supplemented with 6 mM L-Glutamine, 50% DMEM/F-12 (supplemented with 6 mM L-Glutamine), 20% conditioned media and 1× InstiGRO CHO supplement. The media can be serum free and free of animal derived components;
(b) selecting and isolating one or more single cell clones from step (a);
(c) expanding each of the one or more single cell clones from step (b) in a serum-free media;
(d) selecting from each of the one or more single cell clones from step (c) at least one of the following characteristics:
   (i) a cell viability,
   (ii) a cell doubling time,
   (iii) a transfection efficiency,
   (iv) a peak viable cell density; and
   (v) clumping; and
(e) isolating and expanding a single cell selected from step (d) in a serum-free media thereby producing the host cell line.

In some aspects, the host cell is cultured with conditioned medium. Once the culture medium is incubated with cells, it is referred to as “spent” or “conditioned medium”. Conditioned medium contains many of the original components of the medium, as well as a variety of cellular metabolites and secreted proteins, which may include, for example, growth factors, inflammatory mediators, and other extracellular proteins. In one aspect, conditioned medium can be obtained by (a) culturing a host cell derived from the parental cell line at an initial density to produce a second cell culture medium comprising the cells at a higher density and factors secreted by the cultured cells; and (b) separating the cells from the second culture medium to produce the conditioned media.

In some aspects, the host cell is produced by selecting a host cell having at least one of the following characteristics compared to the parental cell line:
(i) improved cell viability;
(ii) improved peak viable cell density;
(iii) improved population doubling time (in hours);
(iv) improved transfection or nucleofection efficiency;
(v) increased expression levels of one or more genes related to immune response;
(vii) increased outgrowth of cells when after seeding at low cell density; and
(vii) increased AAV vector titers.

In some aspects, the transfection efficiency of the host cell can be determined by transiently transfecting the cell with the required components. In some aspects, transfection efficiency is determined by transfection with: one plasmid containing the vector genome, which is an expression cassette with promoter, transgene of interest flanked by ITR, and nucleic acid molecules encoding Rep proteins and Cap proteins that are specific for the desired serotype. In some aspects, the transgene of interest used for assessing transfection efficiency is a reporter protein, such as a green fluorescent protein or mCherry.

In some aspects the production of viral particles in a cell is determined by transiently transfecting the host cell with a plasmid containing the vector genome, which is an expression cassette with (i) promoter and transgene of interest flanked by ITR, (ii) nucleic acid molecules encoding Rep and Cap proteins that are specific for the desired serotype; and (iii) a plasmid (Ad helper) carrying the minimal adenoviral genes required to support AAV replication (e.g. E2, E4 and VARNA). In some aspects, the genes coding for AAV and Ad helper function can be cloned in a single plasmid. In some aspects, a host cell is transfected using one plasmid comprising the AAV vector sequence and encodes the AAV genes (Rep and Cap) required to support AAV genome replication and packaging; and a second plasmid which is the pAdHelper plasmid which encodes the genes from a helper virus genome (e.g., a wild-type Adenovirus 5 (wtAd5) genome) that are required to support AAV gene expression, replication, and packaging. In some aspects a wild-type Adenovirus 5 (wtAd5) is used as the helper. In some embodiments, the Rep and Cap are transcribed from endogenous AAV promoters p5, p19 and p40 or modified versions thereof (e.g., a modified p5 promoter).

In some aspects, the viral particles are produced from a host cell by stably integrating into the host cells nucleic acids for a transgene of interest flanked by AAV ITRs and AAV Rep and Cap proteins, to generate a producer cell line. The producer cell line is then infected with a helper virus, such as a wild-type Ad5 adenovirus, to produce rAAV particles. The titer of rAAV particles produced from the host cell with stable integration of elements required for rAAV generation, is indicative of the host cell's robustness as a candidate for producer cell line generation.

In some aspects, the cell viability or viable cell density is determined after freeze-thawing, shear stress, or combination thereof. In some aspects, shear stress comprises stirring cells in culture medium in a cell culture bioreactor system, (e.g. Ambr® 15) at a stirring rate from about 1500 rpm to about 2000 rpm. In some aspects, the shear stress can be determined by measuring cell viability and cell growth at increasing stirring rate of 1500, 1650, 1800, or 2000 rpm. In some aspects shear stress can be determined by measuring cell viability and cell growth at increasing stirring rate of 1500, 1650, 1800, or 2000 rpm, and dissolved oxygen at 80%. In some aspects, the Ambr® is a table-top microbioreactor system which is a multi-parallel bioreactor enabling testing multiple cell culture conditions in parallel and evaluating cell viability and viable cell density of candidate cell populations under those conditions. Cell viability was measured by Vi-CELL.

A host cell line or producer cell line derived from HeLaS3 parental cell line can have several varying attributes compared to the parent cell line. For example, in some aspects, a host cell or producer cell has about a 0.5% to about a 25% difference (e.g., an increase or a decrease of about 0.5, 1.0, 5.0, 10.0, 15.0, 20.0, 25.0% or more) in cell doubling time compared to the parental cell line (e.g., HeLa S3). In some aspects, a host cell has about a 0.5% to about a 25% difference (e.g., an increase or a decrease of about 0.5, 1.0, 5.0, 10.0, 15.0, 20.0, 25.0% or more) in cell doubling time (in hours) compared to the parental cell line.

In some aspects, a host cell derived from HeLaS3 parental cell line can have a cell doubling time (in hours) compared to the parental cell of less than 32 hours, less than 30 hours, less than 28, hours, less than 26 hours, or less than 24 hours. In some aspects, a host cell derived from HeLaS3 parental cell line can have a cell doubling time (in hours) compared to the parental cell of 30±4 hours, 28±4 hours, 26±4 hours, or less.

In some aspects, a host cell has about a 0.5% to about a 25% difference (e.g., an increase or a decrease of about 0.5, 1.0, 5.0, 10.0, 15.0, 20.0, 25.0% or more) in transfection efficiency compared to the parental cell line.

In some aspects, the transfection is by nucleofection. In some aspects, a host cell has about a 0.5% to about a 25% difference (e.g., an increase or a decrease of about 0.5, 1.0, 5.0, 10.0, 15.0, 20.0, 25.0% or more) in nucleofection efficiency compared to the parental cell line. In some aspects, transfection (e.g. nucleofection) as determined by, e.g., the presence of an intracellular fluorescent protein (e.g. green fluorescent protein), by flow cytometry, or other suitable method. In some aspects, transfection is conducted on cells that have been passaged from 2 to 10 passages post-thaw. In some aspects, the host cell has a transfection efficiency of greater than or equal to 12%. In some aspects, the host cell has a transfection efficiency of greater than 30%. In some aspects, the host cell has a transfection efficiency of 30 to 90%, 40-90% or 60-90%. In some aspects, the host cell has a transfection efficiency of greater than or equal to 30% or 30 to 90%, 40 to 90% or 60 to 90%. In some aspects, transfection efficiency is determined using a reporter transgene such as green fluorescent protein or mCherry.

In some aspects, host cells selected after freeze-thaw, shear stress, or combination thereof have a cell viability greater than or equal to 60%, a cell viability greater than or equal to 70%, greater than or equal to 80%, greater than or equal to 90% or greater than or equal to 95%. In some aspects, the cells selected after freeze-thaw, shear stress, or combination thereof have a cell viability from 70%-99% cell viability. In some aspects, the cells selected after freeze-thaw, shear stress, or combination thereof have a cell viability from 70-99% cell viability after the first selection step, the second selection step, the third selection step or any additional step thereafter. In some aspects, the cell viability is determined by standard viability assays such as the trypan blue dye exclusion method or other suitable method.

In some aspects, a host cell has about a 0.5% to about a 25% difference (e.g., an increase or a decrease of about 0.5, 1.0, 5.0, 10.0, 15.0, 20.0, 25.0% or more) in peak viable cell density compared to the parental cell line.

In some aspects, a host cell has about a 0.5% to about a 25% difference (e.g., an increase or a decrease of about 0.5, 1.0, 5.0, 10.0, 15.0, 20.0, 25.0% or more) in percent cell viability after one cell free thaw cycle compared to the parental cell line.

In some aspects, a host cell has about a 0.5% to about a 25% difference (e.g., an increase or a decrease of about 0.5, 1.0, 5.0, 10.0, 15.0, 20.0, 25.0% or more) in percent cell viability after one round of sheer stress compared to the parental cell line.

In some aspects, a host cell has peak viable cell density of greater than or equal to 3E6 cell/mL. In some aspects, the host has peak viable cell density of 3E6 to 6E6 or 3E6 to 5E6 or 5E6 to 6E6. In some aspects, the host cell has a transfection efficiency of greater than or equal to 12%, 15%, 20%, 30%, 40%, 50%, 60% or more of cells. In some aspects, the host cell has a transfection efficiency of greater than 30%. In some aspects, the host cell has a transfection efficiency of greater than or equal to 30% of cells with a transgene such as green fluorescent protein.

In some aspects, a host cell has reduced clumping or aggregation of cells visible to the naked eye. In some aspects, the host cell has reduced clumping or aggregation of cells visible to the naked eye as determined 3 or 4 days of culture after seeding.

In some aspects, producer cell generated using a host cell isolated from a parental cell line, using methods described herein, has about a 1- to 20-fold difference in AAV vector production titer (vg/mL) (e.g., an increase or a decrease) compared to the parental cell line. In an aspect, AAV vector production can be greater than 1E8, 1E9, or 1E10 vg/mL or more. In some aspects, the method comprises producing an AAV titer of about to about 1E11 AAV vector genomes/cell (vg/cell). In some aspects, the method comprises producing about 1E9 to about 1E10 AAV (vg/mL), about 2E9 to about 6E10 vg/mL, or about 5E9 to about 6E10 vg/mL, or about 1E10 to about 6E10 vg/mL. In some aspects, the vg/mL and vg/cell can be determined by standard methods, including but not limited to—droplet digital PCR (ddPCR) or quantitative polymerase chain reaction (qPCR) of whole cell lysates or purified vector particles, or the like.

In some aspects, a host cell can further be selected by measuring AAV vector production titer and metabolic profiling. In some aspects the host cell's rate of glucose consumption or lactate production or both are evaluated. In some aspects the host cell's rate of change in glucose consumption or lactate production or both are evaluated compared to the parental cell line.

In some aspects, a host cell line is grown in a selection media.

In some aspects, the one or more nucleic acid molecules can be stably transfected into the host cell line.

In some aspects, the selection can further comprise a cell freeze-thaw cycle evaluation, an evaluation of a degree of cell clumping, or combination thereof.

In some aspects, the host cell is selected with the suitable attributes are described in the Examples section and figures.

In some aspects, the producer cell line for production of rAAV particles can be created by the method comprising:
(a) transfecting a host cell line that has been selected with desired characteristics with one or more nucleic acids encoding (i) a heterologous transgene, (ii) an AAV rep gene and an AAV cap gene, (iii) at least one AAV inverted terminal repeat (ITR). The host cell line can infected with a with a helper virus.
   (b) Selecting the producer cell line that generates a titer of rAAV particles of at least about 1E9 to about 1E11 vg/mL. In some aspects, after the transfection and selection process is completed, cells are expanded, and allowed to grow to a higher density (for example to 3.5E5 cells) and further subjected to a primary screen, the primary screen results in measuring the rAAV titer production. In some aspects, the steps of expanding selected cells and further selecting cells for rAAV production titer can be repeated several times until the desired producer cell line is generated. In some aspects, the rAAV titer is determined by quantitative polymerase chain reaction (qPCR).

In some aspects, the method of generating a producer cell line comprises
(a) transfecting a host cell line that has been selected by the method described herein with one or more nucleic acids encoding (i) a heterologous transgene, (ii) an AAV rep gene and an AAV cap gene, (iii) two AAV inverted terminal repeat (ITR). The host cell can be infected with a helper virus.
(b) Selecting the producer cell line where the producer cell line generates a titer of rAAV particles of at least about 1E9 to about 1E11 vg/mL.

In some aspects, the method for generating a producer cell line candidate for production of rAAV particles, comprises the method of:
(a) stably transfecting the host cell line as described herein in serum free medium with one or more nucleic acids encoding (i) a heterologous transgene flanked by two AAV inverted terminal repeats, (ii) an AAV rep gene and an AAV cap gene to generate a producer cell line, and
(b) infecting the producer cell line with an AAV helper virus to produce rAAV particles; and selecting the producer cell line as a candidate for production of rAAV particles if the producer cell line generates a titer of rAAV particles of at least about 1E9 vg/mL.

### Viral Particles

Certain aspects provide methods for producing recombinant adeno-associated virus (rAAV) particles containing rAAV genomes. An rAAV can comprise a heterologous transgene flanked by two AAV inverted terminal repeats (ITRs) packaged into a capsid. A nucleic acid molecule can be encapsidated in the AAV particle. An AAV particle can also comprise capsid proteins. In some aspects, a nucleic acid molecule comprises coding sequence(s) of interest (e.g., a heterologous transgene) operatively linked components in the direction of transcription, control sequences including transcription initiation and termination sequences, thereby forming an expression cassette. An expression cassette can be flanked on the 5′ and 3′ end by two AAV ITR sequences. By “functional AAV ITR sequence” it is meant that the ITR sequence functions as intended for the rescue, replication, and packaging of the AAV particle. See Davidson et al., PNAS, 2000, 97(7)3428-32; Passini et al., J. Virol, 2003, 77(12):7034-40; and Pechan et al., Gene Ther., 2009, 16:10-16, all of which are incorporated herein in their entirety by reference. For practicing some aspects, a recombinant vector comprises at least all of the sequences of AAV essential for encapsidation and the physical structures for infection by the rAAV. AAV ITRs for use in the vectors need not have a wild-type nucleotide sequence (e.g., as described in Kotin, Hum. Gene Ther., 1994, 5:793-801), and can be altered by the insertion, deletion, or substitution of nucleotides or the AAV ITRs can be derived from any of several AAV serotypes. More than 40 serotypes of AAV are currently known, and new serotypes and variants of existing serotypes continue to be identified. See Gao et al., PNAS, 2002, 99(18):11854-6; Gao et al., PNAS, 2003, 100(10):6081-6; and Bossis et al., J. Virol, 2003, 77(12):6799-810. Use of any AAV serotype is considered within the scope of the present application. In some aspects, a rAAV vector is a vector derived from an AAV serotype, including without limitation, AAV1, AAV2, AAV3, AAV4, AAV5, AAV6, AAV7, AAV8, AAVrh8, AAVrh8R, AAV9, AAV10, AAVrh10, AAV11, AAV 12, AAV2R471A, AAV DJ, a goat AAV, bovine AAV, or mouse AAV or the like. For example, in some aspects, the AAV serotype is AAV1, AAV2, AAV5, AAV6, AAV7, AAV8, AAVrh8, AAVrh8R, AAV9, AAV10, or AAVrh10. In some aspects, the nucleic acid molecule in the AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype ITRs or the like. In certain aspects, the nucleic acid molecule in the AAV comprises an AAV2 ITR. In further aspects, rAAV particles can comprise an AAV1 capsid, an AAV2 capsid, an AAV3 capsid, an AAV4 capsid, an AAV5 capsid, an AAV6 capsid (e.g., a wild-type AAV6 capsid, or a variant AAV6 capsid such as ShHIO, as described in U.S. PG Pub. 2012/0164106), an AAV7 capsid, an AAV8 capsid, an AAVrh8 capsid, an AAVrh8R capsid, an AAV9 capsid (e.g., a wild-type AAV9 capsid, or a modified AAV9 capsid as described in U.S. PG Pub. 2013/0323226), an AAV10 capsid, an AAVrh10 capsid, an AAV11 capsid, an AAV12 capsid, a tyrosine capsid mutant, a heparin binding capsid mutant, an AAV2R471A capsid, an AAVAAV2/2-7m8 capsid, an AAV DJ capsid (e.g., an AAV-DJ/8 capsid, an AAV-DJ/9 capsid, or any other of the capsids described in U.S. PG Pub. 2012/0066783), an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid, a rAAV2/HBoVl capsid, or an AAV capsid as described in U.S. Pat. No.8,283,151 or International Publication No. WO/2003/042397. In some aspects, a mutant capsid protein maintains the ability to form an AAV capsid. In some aspects, an rAAV particle comprises AAV5 tyrosine mutant capsid (Zhong L. et al., (2008) Proc Natl Acad Sci U S A 105(22):7827-7832. In further aspects, an rAAV particle comprises capsid proteins of an AAV serotype from Clades A-F (Gao, et al., J. Virol. 2004,78(12):6381). In some aspects, an rAAV particle comprises an AAV1 capsid protein or mutant thereof. In other aspects, an rAAV particle comprises an AAV2 capsid protein or mutant thereof. In some aspects, an AAV serotype is AAV1, AAV2, AAV5, AAV6, AAV7, AAV8, AAVrh8, AAVrh8R, AAV9, AAV10, or AAVrh10. In some aspects, an rAAV particle comprises an AAV serotype 1 (AAV1) capsid. In some aspects, an rAAV particle comprises an AAV serotype 2 (AAV2) capsid. In some aspects, an rAAV particle comprises an AAVrh8R capsid or mutant thereof.

### Heterologous Transgenes

In some aspects, a viral particle is a recombinant AAV particle comprising a heterologous nucleic acid molecule (e.g., a heterologous transgene) flanked by two AAV inverted terminal repeats (ITRs). A heterologous nucleic acid molecule can be encapsidated in the AAV particle. In some aspects, a rAAV genome of the present disclosure contains one or more AAV inverted terminal repeats (ITRs) and a heterologous transgene. For example, in some aspects, a rAAV genome of the present disclosure contains two AAV inverted terminal repeats (ITRs). In certain aspects, a rAAV genome of the present disclosure contains two AAV inverted terminal repeats (ITRs) and a heterologous transgene. In some aspects, the vector genome is between about 4.7 kb and about 10 kb. In some aspects, the vector genome is greater than about 5 kb. In some aspects, the vector genome is between about 5 kb and about 7 kb, between about 4.7 kb and about 9.4 kb, or between about 4.7 kb and 6.7 kb, or any value therebetween. In some aspects, the vector genome is greater than about any of 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.2 kb, 9.3 kb, 9.4 kb or more in length or any value therebetween.

In some aspects, a heterologous transgene encodes a therapeutic transgene product. In some aspects, a therapeutic transgene product is a therapeutic polypeptide. A therapeutic polypeptide can, e.g., supply a polypeptide and/or enzymatic activity that is absent or present at a reduced level in a cell or organism. Alternatively, a therapeutic polypeptide can supply a polypeptide and/or enzymatic activity that indirectly counteracts an imbalance in a cell or organism. For example, a therapeutic polypeptide for a disorder related to buildup of a metabolite caused by a deficiency in a metabolic enzyme or activity can supply a missing metabolic enzyme or activity, or it can supply an alternate metabolic enzyme or activity that leads to reduction of the metabolite. A therapeutic polypeptide can also be used to reduce the activity of a polypeptide (e.g., one that is overexpressed, activated by a gain-of-function mutation, or whose activity is otherwise misregulated) by acting, e.g., as a dominant-negative polypeptide.

In some aspects, a therapeutic transgene product is a therapeutic nucleic acid molecule. In some aspects, a therapeutic nucleic acid molecule may include without limitation an siRNA, an shRNA, an RNAi, an miRNA, an antisense RNA, a ribozyme or a DNAzyme. As such, a therapeutic nucleic acid molecule can encode an RNA molecule that when transcribed from the nucleic acid molecules of the vector can treat a disorder by interfering with translation or transcription of an abnormal or excess protein associated with the disorder. For example, a heterologous transgene can encode an RNA molecule that treats a disorder by highly specific elimination or reduction of mRNA encoding the abnormal and/or excess proteins. Therapeutic RNA sequences include RNAi, small inhibitory RNA (siRNA), micro RNA (miRNA), and/or ribozymes (such as hammerhead and hairpin ribozymes) that can treat disorders by highly specific elimination or reduction of mRNA encoding the abnormal and/or excess proteins.

An aspect provides methods for producing a recombinant adeno-associated virus (rAAV) particle containing a recombinant AAV genome using producer cell lines. In some aspects, methods for generating rAAV are provided. Generating rAAV can include (a) transfecting a selected host cell (e.g., a host cell generated via the methods described herein) with a nucleic acid molecule encoding AAV rep and cap genes and a nucleic acid molecule encoding a transgene flanked by two ITRs. This results in a producer cell line. rAAV can then be produced by infecting the producer cell line with AAV helper functions (e.g., an Ad5 helper virus or other suitable helper virus) to generate rAAV. In some aspects, an rAAV genome is between about 4.7 kb and about 10 kb. In other aspects an AAV genome is between about 4.7 kb and 5.1 kb.

In some aspects, the nucleic acid molecule encoding AAV cap is derived from AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV V708K, a goat AAV, AAV1/AAV2 chimeric, bovine AAV, or mouse AAV capsid rAAV2/HBoV1 or variants thereof. In some aspects, the AAV helper is an AAV helper virus or vector. In some aspects the host cell is infected with a helper virus. In some aspects, the helper virus is adenovirus, herpes simplex virus, vaccinia virus, or cytomegalovirus.

In some aspects, the helper is an adenovirus type 5 of subgroup C (Ad5). In some aspects, the Ad5 helper uses the CAR receptor for uptake into the cell.

An adenovirus helper (Ad) is a small non-enveloped virus. The Ad genome encodes approximately 39 genes, which are classified as either early or late depending on whether they are expressed before or after DNA replication. Helper functions for AAV expression are provided by early Ad transcription units encoding proteins E1A, E1B, E2A, and E4, and transcription unit VA RNA. Major late proteins are organized in the transcription units L1 to L5.

In certain embodiments, helper nucleic acid molecules or viruses include Ad nucleic acid molecules. A helper nucleic acid molecule can include one or more of genes E1A, E1B, E2, E3, E4, encoding proteins IX and IVa2, regions L1-L5, and virus-associated (VA) RNAI and VA RNAII, or fragments thereof. Ad helper nucleic acid molecules can be derived from any hAd type, such as Ad5 or Ad2. In certain embodiments, an Ad nucleic acid molecule comprises Ad5 genes. In certain aspects, a helper nucleic acid molecule or virus can encode one or more of Ad E4, E2A, VA RNA, or fragments thereof. Adenovirus helper genes can be present in a vector, in a helper adenovirus, or integrated into a cell genome.

In some aspects, the producer cell line comprises stably maintained nucleic acid molecule encoding AAV rep and cap genes. In some aspects, AAV replication and/or capsid genes are stably maintained in the producer cell line. In some aspects, an AAV vector genome comprising one or more, such as two AAV ITRs and heterologous nucleic acid molecule (e.g., a heterologous transgene) are stably maintained in the producer cell line. In some aspects, AAV replication and/or capsid genes and an AAV vector genome comprising one or more such as two AAV ITRs and heterologous nucleic acid molecule (e.g., a heterologous transgene) are stably maintained in the producer cell line. In some aspects, one or more of AAV replication genes, capsid genes, or an AAV vector genome comprising one or more, such as two AAV ITRs are stably integrated into the genome of the producer cell line. Stably maintained nucleic acid molecules are maintained in the producer cell line upon multiple passages (e.g., 5, 10, 15, 25, or more passages).

In some aspects, an AAV vector genome comprising one or more, such as two AAV ITRs and heterologous nucleic acid molecule (e.g., a heterologous transgene) are transiently transfected in the host cell. In some aspects, an AAV vector genome comprising one or more such as two AAV ITRs and heterologous nucleic acid molecule (e.g., a heterologous transgene) are stably transfected in the host cell line.

In some aspects, the recombinant AAV (rAAV) particle produced by a method comprising:
a) culturing a host cell line under conditions to generate the rAAV particles, wherein the AAV producer cell line comprises
   i) a nucleic acid molecule encoding the AAV rep and cap which are integrated into the genome of the AAV producer cell line,
   ii) a nucleic acid molecule encoding a heterologous transgene flanked by inverted terminal repeats (ITRs), and
   iii) a nucleic acid molecule encoding a selectable marker;
b) providing a AAV helper virus or a derivative thereof; and
c) harvesting the rAAV particles.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the aspects described in broad terms above.

### Example 1: Generation of Host Cell Derived From a HeLaS3 Parental Cell Line

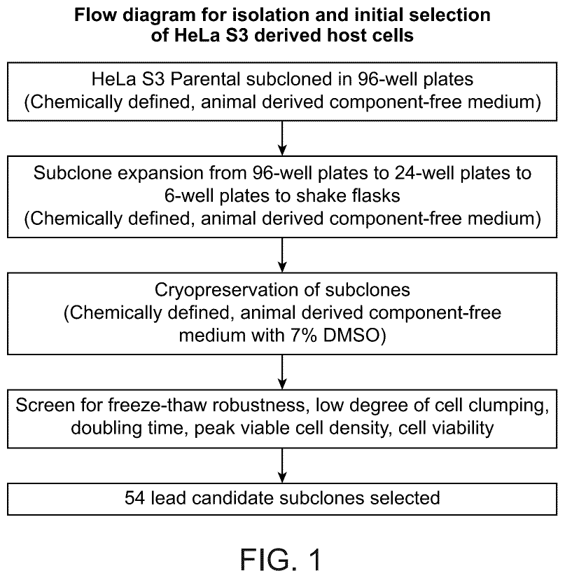

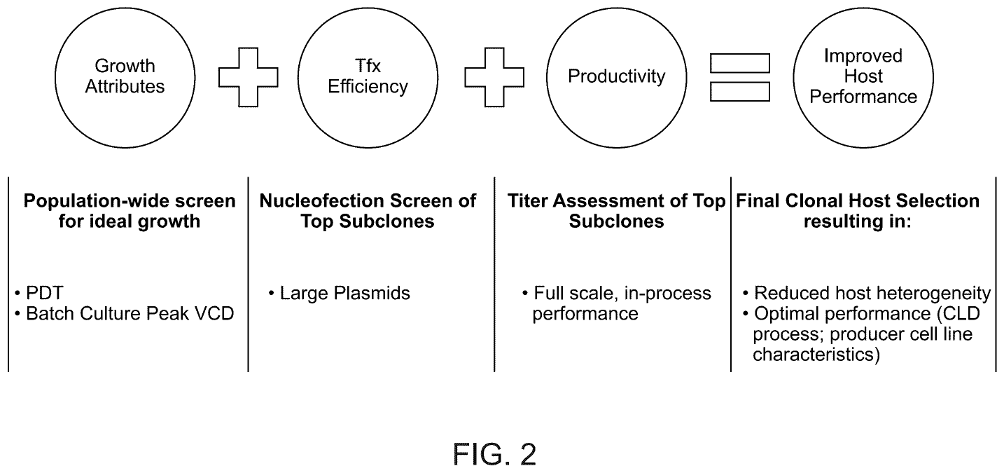

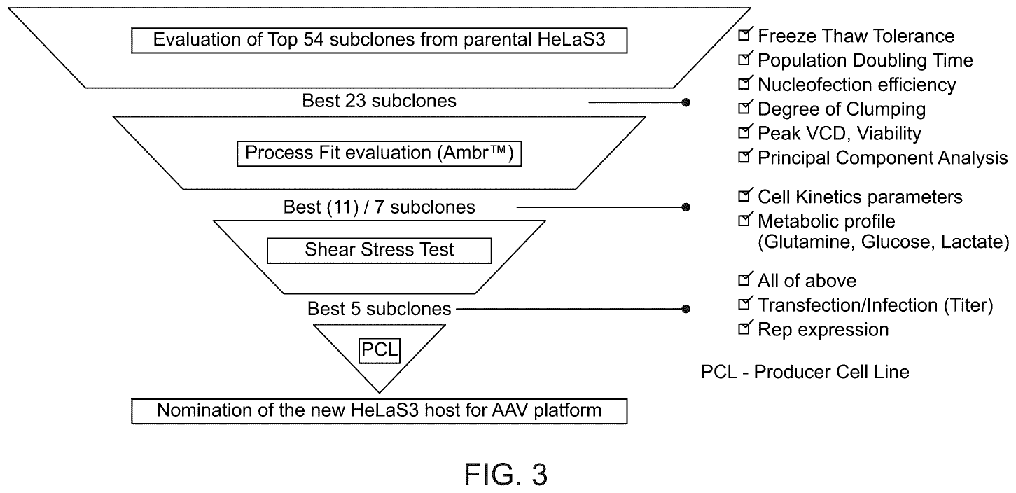

To derive improved host cell lines a methodology as shown in FIG. 1 was established. First, the HeLaS3 parental line was subcloned into 96-well plates to achieve a seeding density of ≤1 cell per well. For wells with clonally derived cell growth, the cells were expanded to 24-well plates, then to 6-well plates, then to shake flasks. Each subclone was cryopreserved into multiple cryovials. Subclones were thawed and screened for freeze-thaw robustness (including low degree of clumping) and cell growth properties, resulting in 54 lead candidate subclones for further evaluation. The schematic in FIG. 2 summarizes some of the favorable cell attributes present in the improved host cell lines.

Using the screen methodology, 54 distinct candidate cell populations were generated from the HeLaS3 parental cell line subcloning. These populations were then narrowed to 23 candidate cell populations based on parameters such as freeze thaw tolerance, population doubling time, transfection efficiency, degree of cell clumping, peak VCD. The 23 candidate cell populations were then assessed in a secondary screen culturing the 23 candidate cell populations in an automated bioreactor system (sheer stress test) which allowed for a further narrowing to 5 candidate cell populations. Finally, the remaining 5 candidate cell populations were stably transfected with components necessary for rAAV production, as described herein, and then infected with a helper virus and assessed for their ability to produce high viral titers.

### Conclusion

From the remaining 5 candidate cell populations the top candidate cell populations remaining were individually cultured and frozen. These top candidate cell populations were selected as the host cell lines are better suited for large scale rAAV production and more amenable to cGMP process development than the HeLa parental cell line. The phenotypic profiling of each host cell line can be established by the screening methods described herein can be complemented by genome sequencing analysis.

### Example 2—Initial Screen to Identify Improved Host Cell Lines

### Introduction

As described in Example 1, a screening approach was used to identify improved host cell lines. This Example focuses on the results obtained from the initial screening process performed on thirty-two of the candidate cell populations. The relative production screen included an evaluation of (i) freeze-thaw tolerance, (ii) cell doubling time, (iii) transfection efficiency, and (iv) assessment of the peak viable cell density. All cell culture media used in these assessments were serum free.

### Results

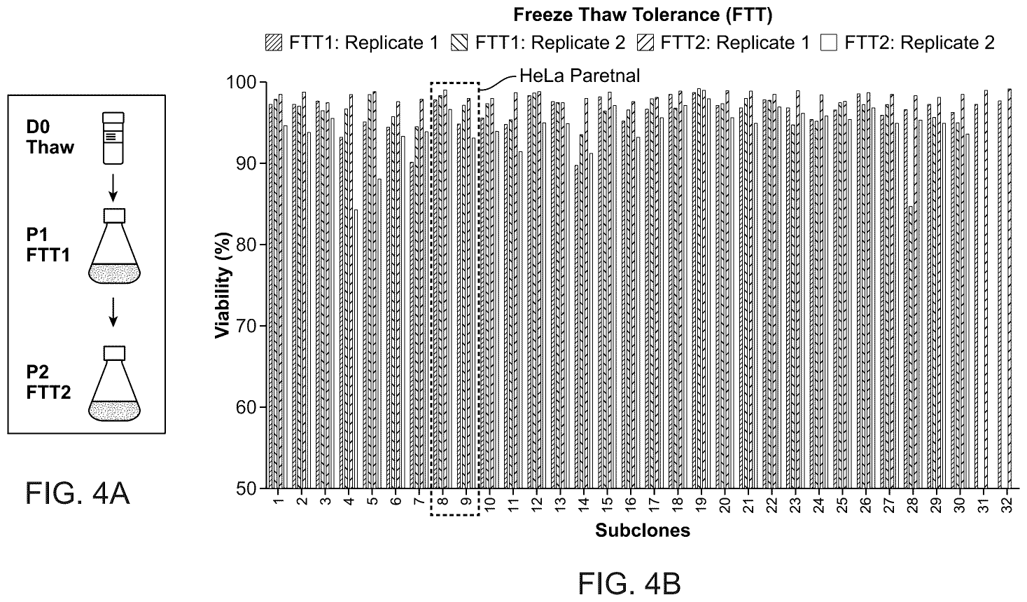

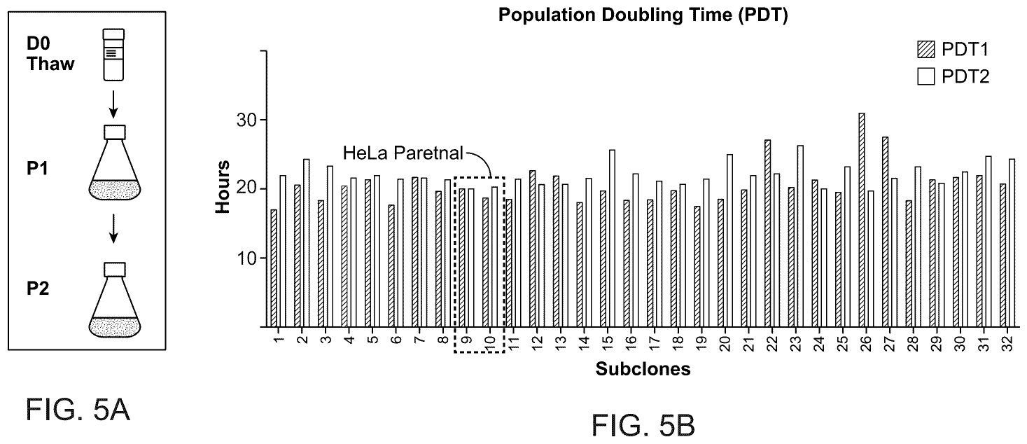

Freeze-thaw tolerance (FTT) was evaluated by thawing thirty-two frozen candidate cell populations, growing them in liquid culture, and assessing their viability after two passages. The FTT experimental set-up is outlined in FIG. 4A. As shown in FIG. 4B, most of the candidate cell populations demonstrated favorable freeze-thaw tolerance with over 90% cell viability.

Population doubling time (PDT) was assessed by monitoring the rate at which the candidate cell populations doubled over approximately 30 hours. The PDT experimental set-up is outlined in FIG. 5A. As shown in FIG. 5B, the rate of PDT was similar to or slightly lower when compared to the parental HeLa cell line.

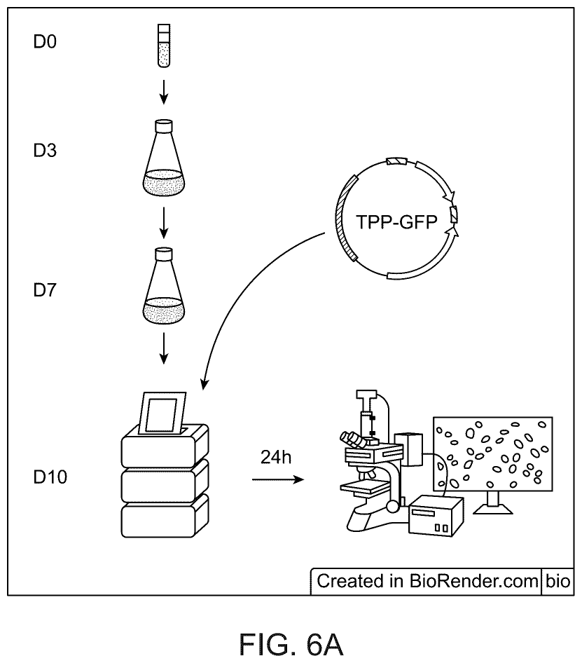

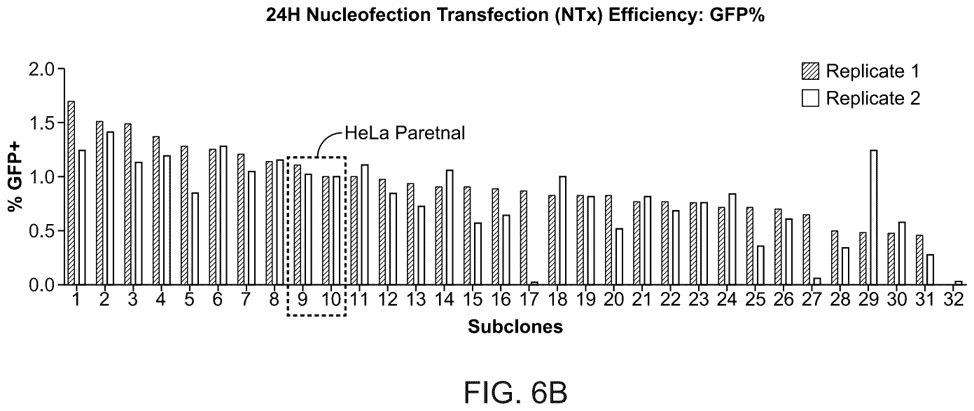

Transfection efficiency was evaluated by transiently transfecting the candidate cell populations with plasmids containing a fluorescent reporter (GFP). The transfection efficiency experimental set-up is outlined in FIG. 6A. As shown in FIG. 6B, there was variation in the rate of transfection efficiency as expressed in percent of GFP positive cells with about half of the candidate populations exhibiting a decreased amount of GFP positive cells when compared to the parental HeLa S2 cell population.

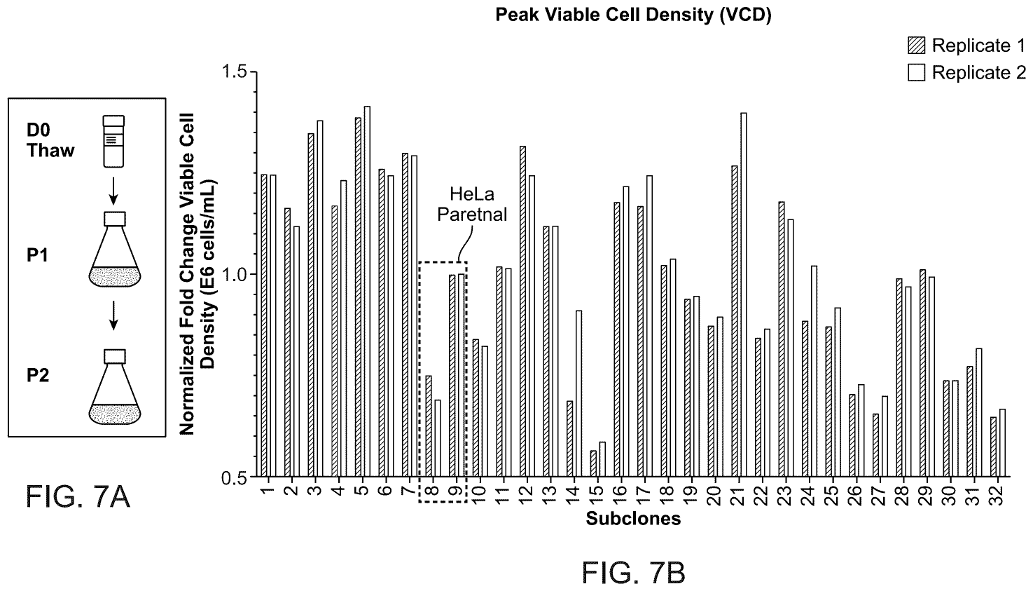

Peak viable cell density (VCD) was measured for the candidate cell populations on over time. The VCD experimental set-up is outlined in FIG. 7A. As shown in FIG. 7B, the viability cell density of the candidate populations greatly varied relative to each other as well as when compared to the parental HeLa cell line. Table 1 lists the subclone IDs for the variants depicted in FIGS. 4B, 5B, 6B, and 7B.

TABLE 1

| Serial Number | Subclone ID |
|---|---|
| 1 | Subclone A |
| 2 | Subclone B |
| 3 | Subclone C |
| 4 | Subclone D |
| 5 | Subclone E |
| 6 | Subclone F |
| 7 | Subclone G |
| 8 | Subclone H |
| 9 | Subclone I |
| 10 | Subclone J |
| 11 | Subclone K |
| 12 | Subclone L |
| 13 | Subclone M |
| 14 | Subclone N |
| 15 | Subclone O |
| 16 | Subclone P |
| 17 | Subclone Q |
| 18 | Subclone R |
| 19 | Subclone S |
| 20 | Subclone T |
| 21 | Subclone U |
| 22 | Subclone V |
| 23 | Subclone W |
| 24 | Subclone X |
| 25 | Subclone Y |
| 26 | Subclone Z |
| 27 | Subclone AA |
| 28 | Subclone BB |
| 29 | Subclone CC |
| 30 | Subclone DD |
| 31 | Subclone EE |
| 32 | Subclone FF |

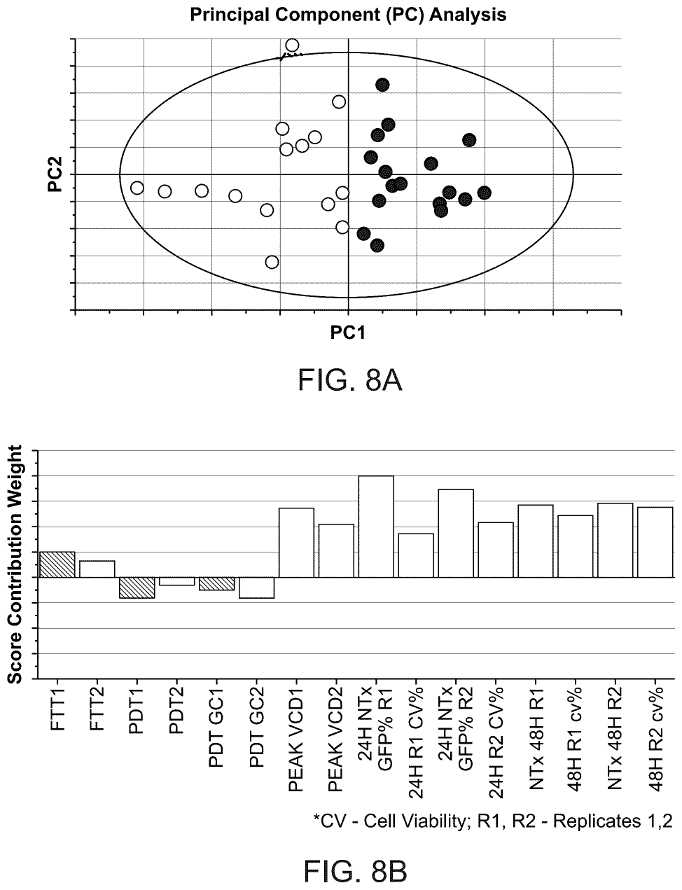

A principal component analysis (PCA) was carried out to assess which of the above evaluations resulted in the most variation. An exemplary resulting PCA biplot for all thirty-two candidate populations (represented by individual dots) is shown in FIG. 8A. As shown in FIG. 8B, the score contribution weights for transfection efficiency and the peak VCD evaluation had the most sample variation.

Collectively, the results from the initial cell performance assessments indicate that several evaluation parameters are required during an initial screen because each candidate cell population displayed a unique array of phenotypic characteristics especially as it relates to their peak VCD and transfection efficiency.

### Example 3—Screening to Identify Improved Host Cell Lines

### Introduction

In Example 2, the results of the initial cell performance assessments narrowed down the original fifty-four candidate cell populations to twenty-three. This Example describes the further evaluations to narrow the candidate cell populations. These evaluations were performed using serum-free media which more closely comply with GMP guidelines. See generally article “Serum-free media: ask the experts” (published in 2022 and available at regmednet.com/serum-free-media-ask-the-experts/). During the second stage, the candidate cell populations underwent a series of assays that aimed to mimic large-scale cell culturing conditions and to specifically evaluate the ability of the host cell line to establish subsequent AAV producer cell lines. Schulze et al. (2021), J. Biotechnol., vol. 335:65-75.

### Results

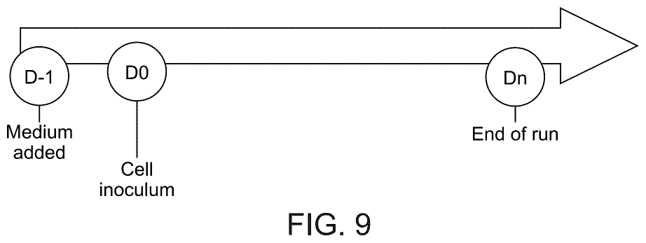

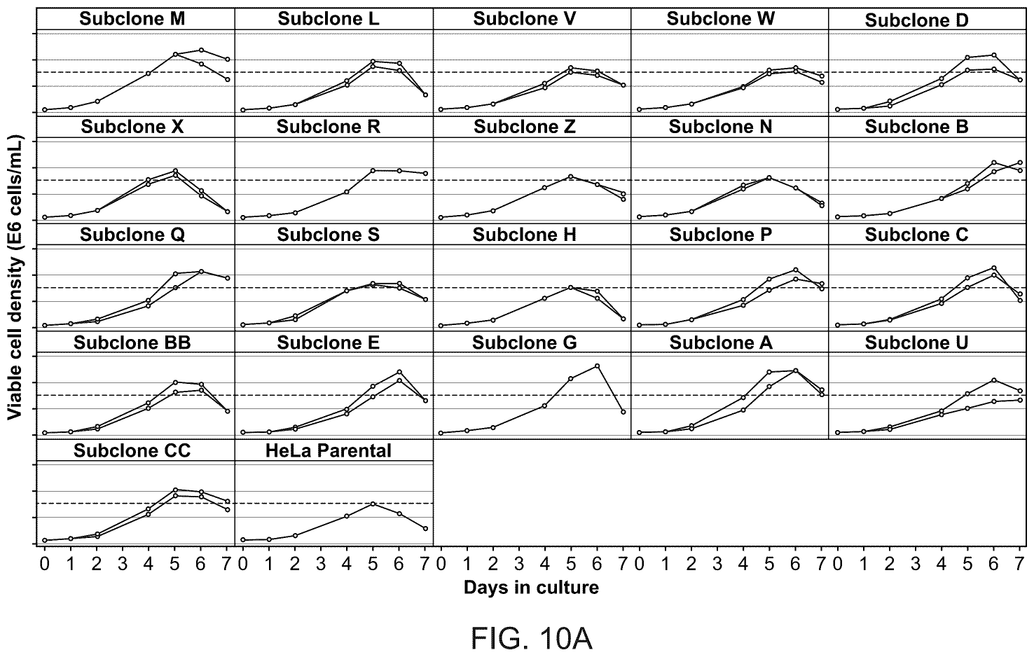

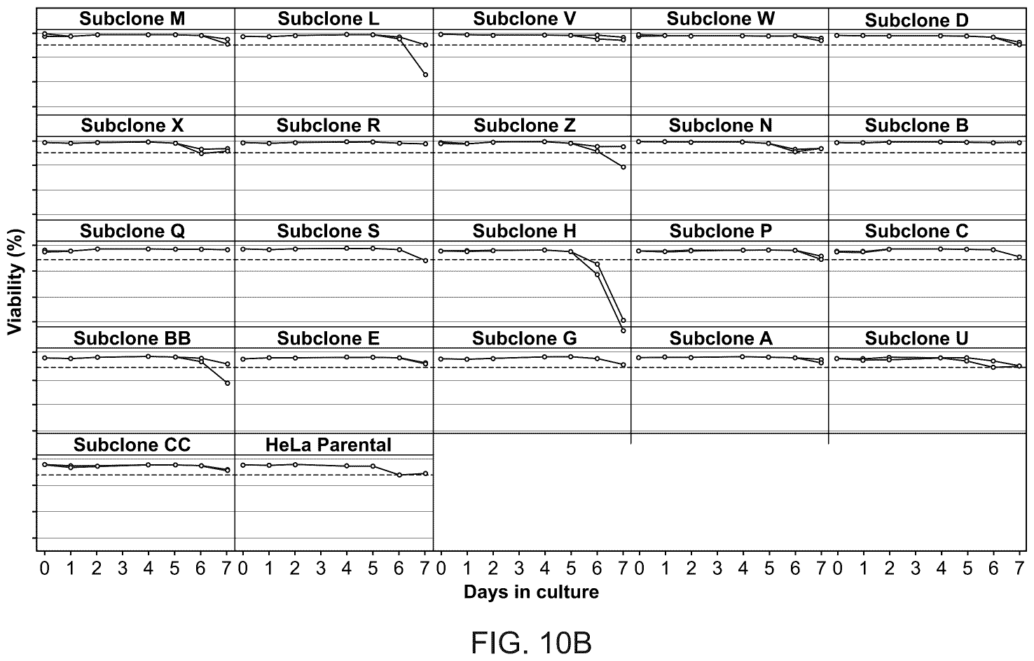

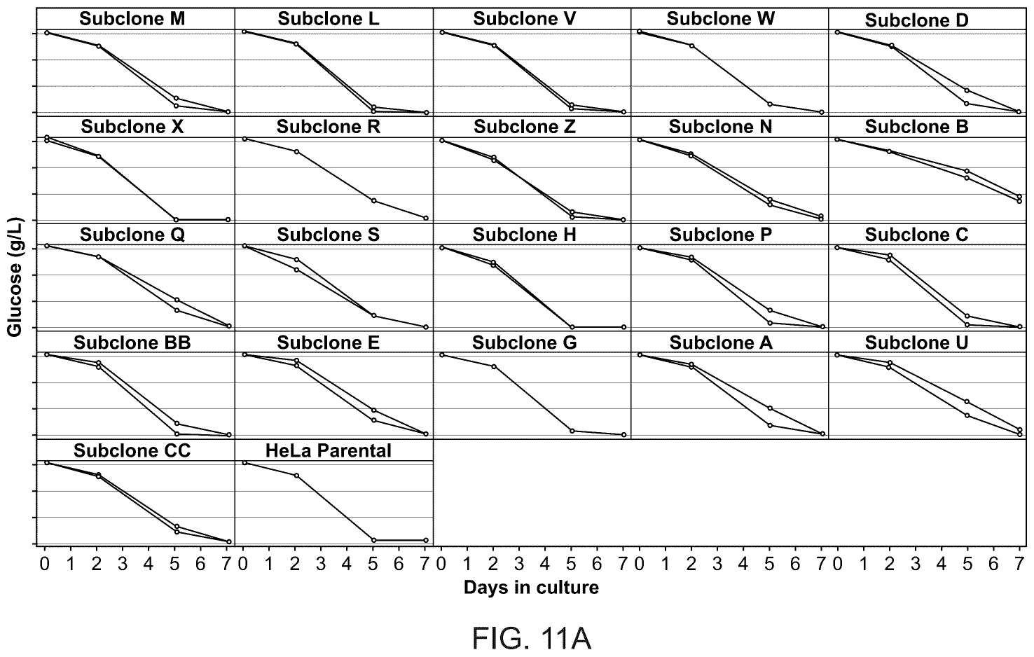

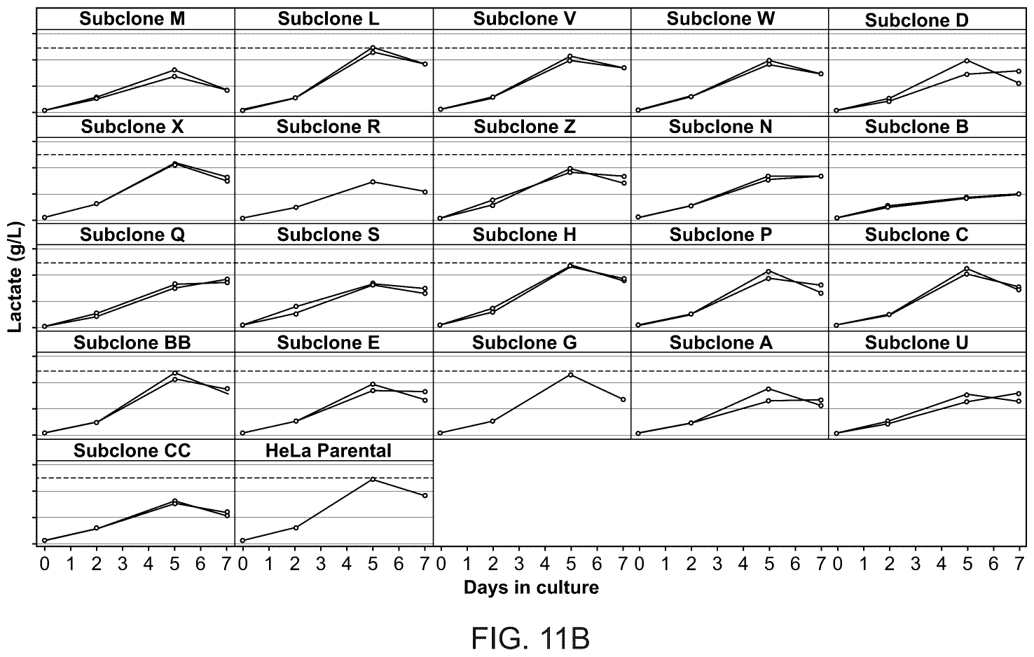

To evaluate the cell growth conditions and metabolic profiling of the twenty-three candidate cell populations, they were cultured in a table-top small-scale microbioreactor system as shown in FIG. 9A. Over a 7-day period, twenty-one of the candidate cell populations were evaluated for their growth conditions (see FIG. 10A) and metabolic profiling (see FIG. 11A-B). As demonstrated in FIG. 10A and FIG. 10B, most of the candidate cell populations exhibited a peak in viable cell density and high cell viability, respectively. However, certain candidate cell populations such as subclone B did not display a VCD peak (as seen in FIG. 10A), and subclone H showed a dramatic reduction in percent viability by day 5 of culturing (as seen in FIG. 10B). As shown in FIG. 11, the glucose depletion (FIG. 11A) and lactate production (FIG. 11B) curves there was variation in the metabolic profiles for each of the candidate cell populations when compared to each other and the HeLaS3 parental cell line.

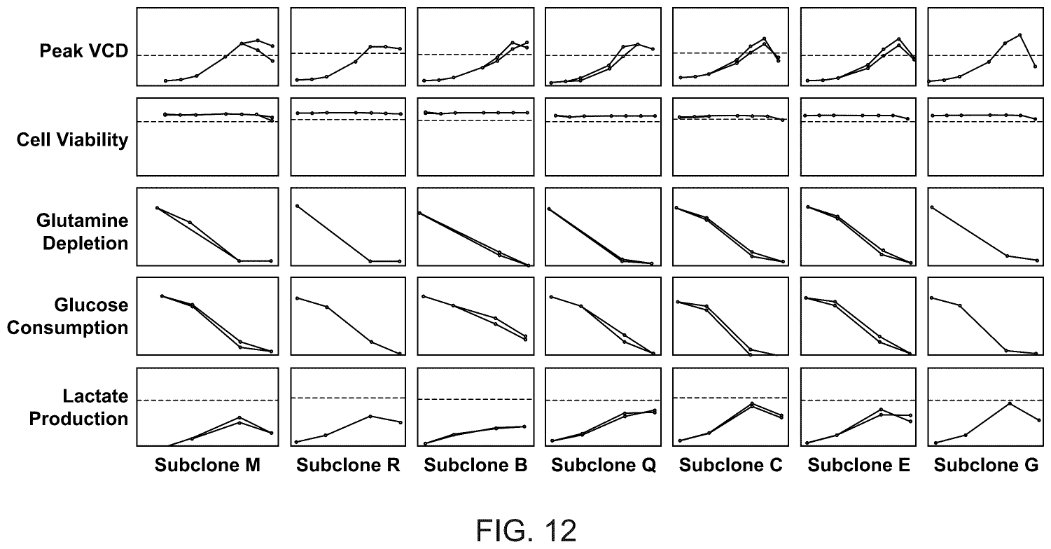

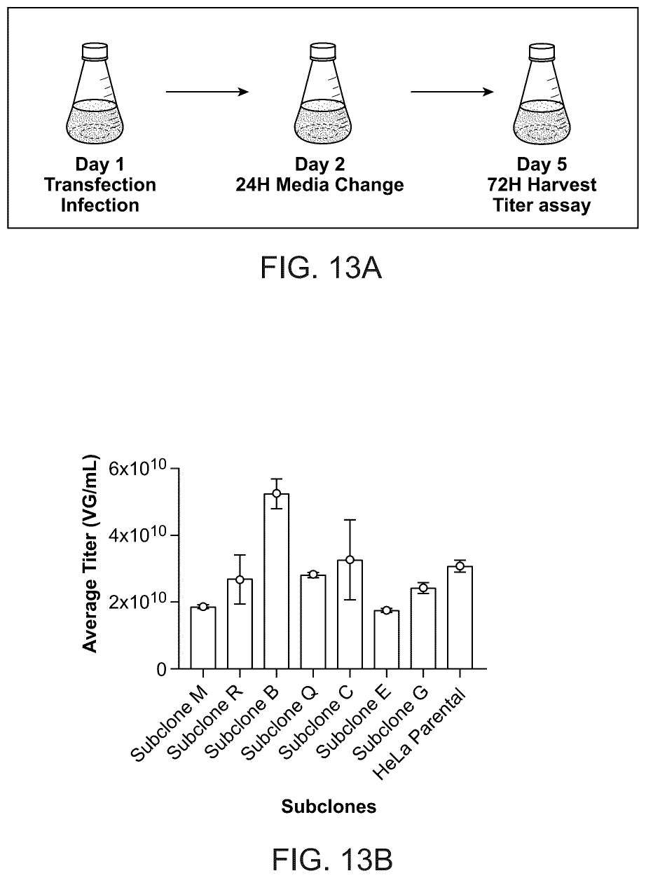

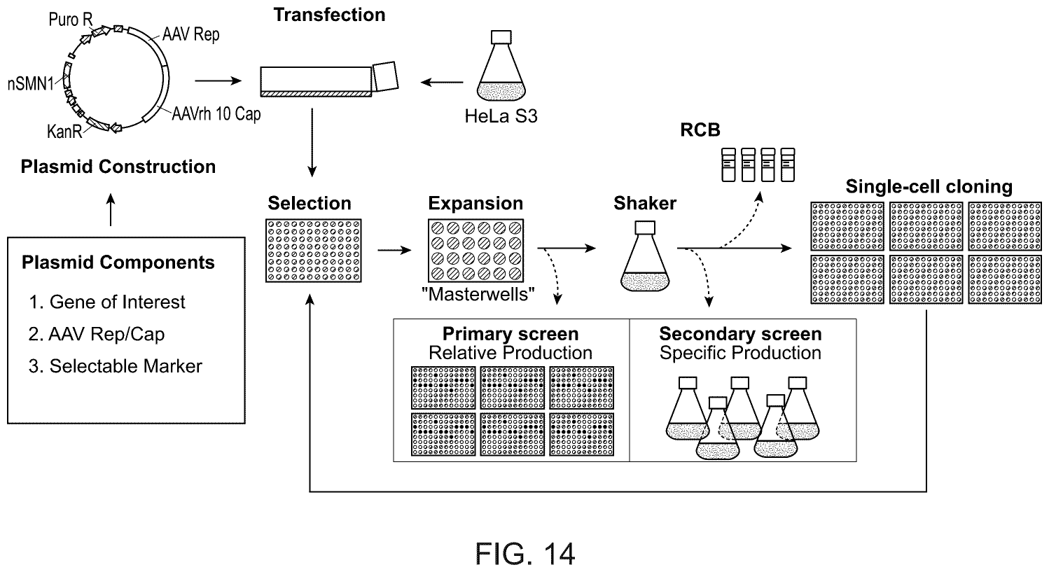

Based on the results of the kinetics and metabolic profiling evaluations, the top seven candidate cell populations were analyzed for their specific ability to produce AAV titer. Seventy-two hours after the transfection with a viral plasmid AAV titer released into the cell culture media was harvested and measured for each candidate as depicted in FIG. 13A. As shown in FIG. 13B, the average viral titer for each of the candidate cell populations varied with the cell populations labeled subclone B and C having the highest viral titers when compared to the HeLaS3 parental control.

Taken together, the results suggest that screening in an Ambr®15 can lead to additional reduction in candidate cell populations. However, it is important to consider several parameters simultaneously because each candidate cell population has its own unique profile and can exhibit variability.

### Example 4: Generation of Host Cell Derived From a HeLaS3 Parental Cell Line

A HeLaS3 parent cell line was thawed, subcultured (in an orbital shaker platform set at 37° C., 5% CO₂, 125 rpm, with a 25 mm orbital diameter) and expanded in medium that was free of any animal derived components (EX-CELL® HeLa growth medium supplemented with 6 mM L-Glutamine). The cells were subcultured for two passages at a density of 0.2×10⁶ cells/mL after thawing. At the third passage, cells were used to prepare (i) Conditioned Medium (CM); and (ii) the culture for limited dilution cloning (LDC) referred to as the N-1 culture. The CM is a medium used to enhance cell outgrowth.

To generate single cell subclones, limiting dilution cloning (LDC) was used. For the LDC process an N-1 culture was first prepared where the HeLaS3 cells were seeded in 125 ml shake flasks at a density of 0.2×10⁶ cells/mL. The cell culture was maintained on an orbital shaker platform set at 125 RPM with a 25 mm orbital diameter at 37° C., 5% CO₂ for 3 days.

For the LDC process, cells with viability exceeding 90% and population doublings less than 26 hours were seeded in a 96-well plate at a density of 0.5 cells/well with EX-CELL® HeLa (supplemented with 6 mM L-Glutamine), 50% DMEM/F-12 (supplemented with 6 mM L-Glutamine), 20% CM and 1× InstiGRO CHO supplement. This medium was serum free and animal derived cell-free except for the EX-CELL® HeLa media component (cod liver oil) that posed no biosafety or TSE/BSE risk.

Throughout the process, cells were imaged at days 0, 4, 7, and 12 to assess clonality (determining that a cell line originated from a single cell).

When colonies were large enough and cells looked healthy, about 21 days after seeding, cells were ready to expand. Cells were sequentially expanded to 24 well plates and 6-well plates, and finally transferred to 125 mL shake flasks. Expansion to 12 mL shake flasks was considered passage 0 for the newly generated subclones.

The subclones were expanded for 2 or 3 passages in suspension culture, after which selected clones were banked in EX-CELL® HeLa media with 6 mM L-Glutamine and 10% DMSO followed by freezing 4 cryovials of each subclone, with 10 million cells per vial. These cryopreserved banked clones were referred to as the Pre-Master Cell Bank (MCB) generation.

To prepare the MCB, subclones generated and cryopreserved as pre-MCB, were thawed. One vial of each subclone was amplified. The thawed clones were expanded during 3 passages in EX-CELL® HeLa growth medium supplemented with 6 mM L-Glutamine. After the third passage, cells were banked in EX-CELL® HeLa growth medium supplemented with 6 mM L-Glutamine and 10% DMSO. 10 million cells/cryovial were frozen in a total to 14 cryovials per clone.

### Example 5—Clone Selection Process Under Serum-Free Conditions

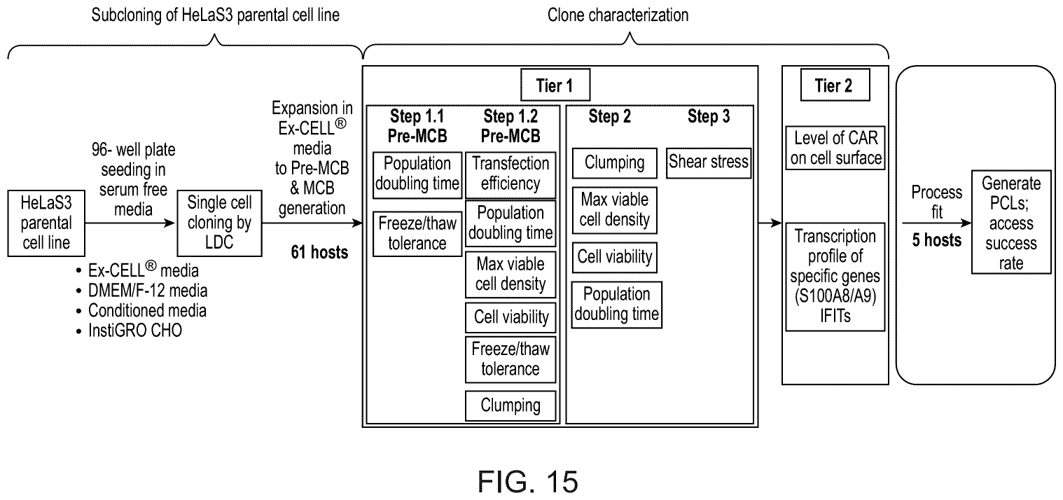

Based on Example 4, 61 clones were identified for further selection, characterization, to ensure consistency and to improve host cell lines. The 61 clones were characterized based on the process shown in FIG. 15.

Briefly, the process was divided into a series of screening or characterization steps.

In the first screening step, Tier 1, the clones were tested for:

### Step 1.1 Characterization in Shake Flask Culture System

a freeze-thaw tolerance, and
b. population doubling time (PDT) in shake flask culture system.

### Step 1.2 Characterization in Shake Flask Culture System

a. Freeze-thaw tolerance
b. PDT and maximum viable cell density (max VCD)
c. Transfection efficiency and cell viability after nucleofection with a triple play plasmid (to produce rAAV2)

### Step 2 Characterization of the Selected Top 23 Clones in a High Throughput Cell Culture Bioreactor System Known as Ambr®15

a. PDT and Max VCD
b. Clumping

### Step 3 Characterization of the Selected Top 11 Clones (Ambr®15)

a. Shear stress tolerance

In a second screening (Tier 2), clones were characterized regarding:
1. Coxsackie-Adenovirus Receptor (CAR) expression levels (11 clones)
2. Immune-related gene expression (7 clones)

Step 1.1 Freeze-thaw tolerance (FTT) was evaluated by thawing the 61 frozen candidate cell populations, growing them in liquid culture, and assessing their viability. PDT was assessed by monitoring the time rate at which the candidate cell populations doubled. PDT was monitored after 3 passages and the average of the PDT after passages 2 and 3 was used to identify relevant clones. Cell viability after thawing was measured by Vi-CELL.

Clones that exhibited a PDT greater than 32 hours or a cell viability after thawing lower than 70% (i.e., about 20% less than cell viability before freezing) or a combination thereof were excluded.

Based on the freeze-thaw and PDT, and the attributes shown in Table 2, 54 clones were selected and were carried on to the next characterization step 1.2.

TABLE 2

| Attribute | Value |
|---|---|
| Freeze-thaw tolerance P0 | Greater than or equal to 70% cell viability |
| Freeze-thaw tolerance P1 | Greater than or equal to 70% cell viability |
| Freeze-thaw tolerance P2 | Greater than or equal to 70% cell viability |
| Freeze-thaw tolerance P3 | Greater than or equal to 70% cell viability |
| PDT Average for P2 and P3 | Less than or equal to 32 hours |

### Step 1.2 Characterization in Shake Flasks

Freeze-thaw tolerance (FTT) was evaluated by thawing the 54 frozen candidate cell populations, growing them in liquid culture (EX-CELL® HeLa growth medium supplemented with 6 mM L-Glutamine) and assessing their viability. PDT was assessed by monitoring the time at which the candidate cell populations doubled their cell concentrations. Cells were sub-cultured every 3 days during 3 passages and both VCD and cell viability were measured.

PDT was monitored after 3 passages and the average of the PDT after passage 2 and 3 were used to identify relevant clones. Cell viability after thawing was measured by Vi-CELL. Cells were sub-cultured every 3 days during 3 passages and both VCD and cell viability were measured.

Clumping was another attribute evaluated. It was noticed that cell aggregates were more visible in the first day of culture and in general, disappeared over the days. Therefore, the clumping observation was made with the naked eye at days 3 and day 4 of culture, after seeding at 0.2×10⁶ cells/mL.

The clumping evaluation in those two consecutive days of culture was done by two operators and the average of all measurements (n=4) was used to evaluate the clones for clumping. A qualitative score (1 to 5) was given by each operator in each day, where:
The relative value of 1 corresponded to many aggregates compared to HeLaS3 parental.
The relative value of 5 corresponds to no aggregates compared to HeLaS3 parental.
HeLaS3 parental cells were used as control and classified with a relative value of 4.
Selected clones studied were classified with a relative value between 2 and 5.

### Cell Growth Curve and Max VCD

On 3rd passage after cell thawing, a growth curve was performed in duplicate. Cells were seeded at 0.2×10⁶ cells/mL and evaluated daily for VCD and cell viability with the Vi-CELL. PDT in the exponential growth phase and max VCD was calculated. Parental HeLaS3 cells were used as a control in all experiments.

### Transfection Efficiency

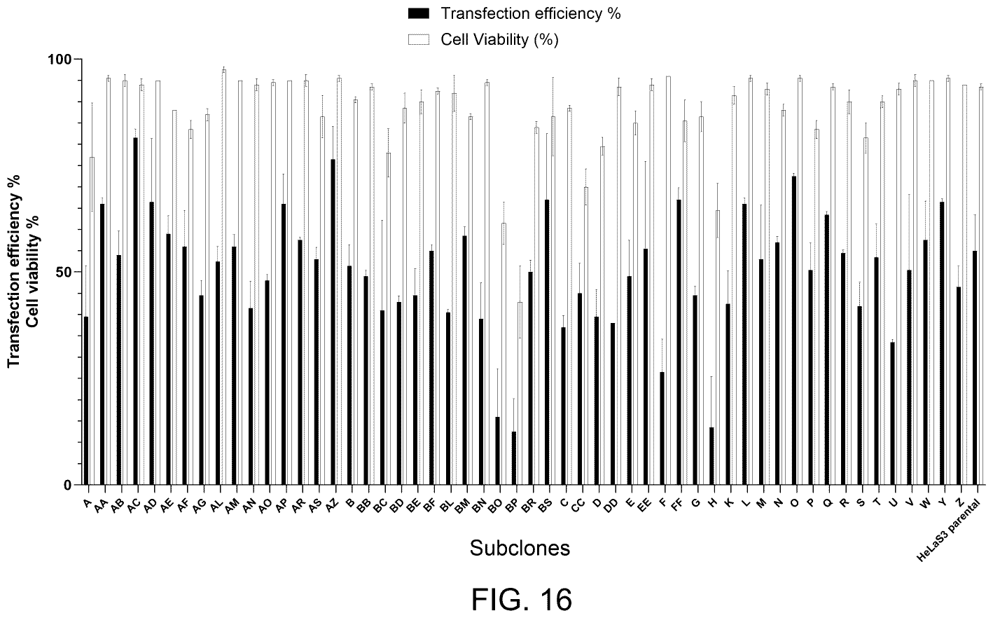

Cells in the exponential growth phase were transfected with pTPK (thiamin pyrophosphokinase 1) Herpes simplex virus (HSV), TK (thymidine kinase), ITR (inverted terminal repeat), CBA (chicken beta actin promoter), enhanced green fluorescent protein (EGFP) plasmid. Forty-eight hours post-transfection, transfection levels were measured by GFP expression by flow cytometry (by BD FACSCELESTA™ SORP flow cytometer) and cell viability by Vi-CELL. The experiment was carried out with two replicates (FIG. 16). In FIG. 16 the transfection efficiency was represented as a percentage of GFP (solid black bars) and the cell viability (white bars) of the 54 clones compared to the HeLaS3 parental cells.

From the 54 cones, 23 clones were selected to continue to the next characterization step (Step 2) based on the attributes shown in Table 3 below.

TABLE 3

| | |
|---|---|
| Attribute | Value |

Performance after thawing

| | |
|---|---|
| Freeze-thaw tolerance P0 | Greater than or equal to 90% cell viability |
| Freeze-thaw tolerance P1 | Greater than or equal to 90% cell viability |
| Freeze-thaw tolerance P2 | Greater than or equal to 90% cell viability |
| PDT P2 | Less than or equal to 32 hours |

Growth Curve

| | |
|---|---|
| PDT Growth Curve Rep 1 | Less than or equal to 32 hours |
| PDT Growth Curve Rep 2 | Less than or equal to 32 hours |
| Peak VCD Rep 1 | Greater than or equal to 3 × 10⁶ cell/mL |
| Peak VCD Rep 2 | Greater than or equal to 3 × 10⁶ cell/mL |
| Clumping | Value = 3 |

Transfection Efficiency

| | |
|---|---|
| % GFP Rep 1 | Greater than or equal to 30% positive cells |
| % GFP Rep 2 | Greater than or equal to 30% positive cells |
| % Viability Rep 1 | Greater than or equal to 60% positive cells |
| % Viability Rep 2 | Greater than or equal to 60% positive cells |

Some clones were selected to continue to the next characterization step despite presenting cell viability below 90% at P1 after thawing if they showed higher cell viability on P2.

### Step 2—Cell Culture on Ambr®15 System

### Cell Growth Performance

The 23 clones with the best performance in Step 1.2 and based on the attributes selected from Table 2 were thawed and expanded for 2 passages. HeLaS3 parental cells were used as control. After two passages, clones were seeded in Ambr®15 with the following conditions:
1. Seeding=0.2×10⁶ cells/mL
2. pH control at 7.2±0.05
3. Constant stirring rate 1500 rpm, downstir
4. Dissolved Oxygen (DO)=50%
5. Antifoam: Foam Away diluted 1:10 added daily

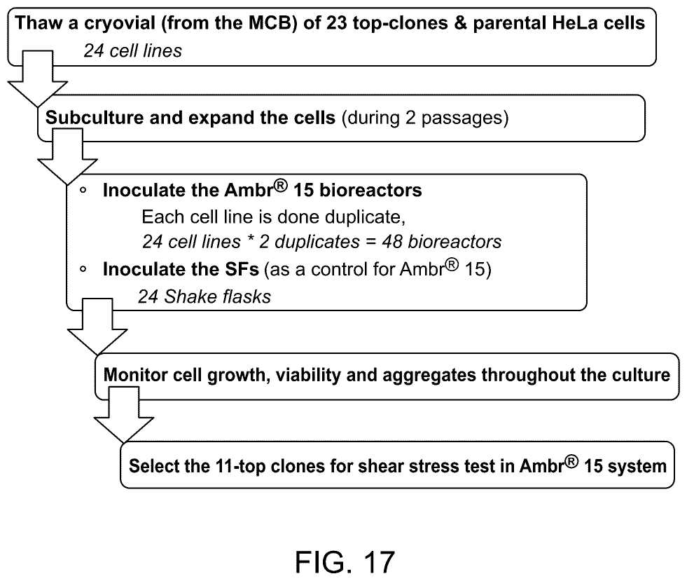

The experimental plan for Step 2 is shown in FIG. 17.

Clones could show a superior performance in some attributes, but minimal level of performance was required in all attributes as shown in Table 4.

TABLE 4

| | |
|---|---|
| Attribute | Value |

Performance after thawing

| | |
|---|---|
| Freeze-thaw tolerance P0 | Greater than or equal to 90% cell viability |
| Freeze-thaw tolerance P1 | Greater than or equal to 90% cell viability |
| Freeze-thaw tolerance P2 | Greater than or equal to 90% cell viability |
| PDT P2 | Less than or equal to 32 hours |

Growth Curve

| | |
|---|---|
| PDT Growth Curve Rep 1 | Less than or equal to 32 hours |
| PDT Growth Curve Rep 2 | Less than or equal to 32 hours |
| Peak VCD Rep 1 | Greater than or equal to 3 × 10⁶ cell/mL |
| Peak VCD Rep 2 | Greater than or equal to 3 × 10⁶ cell/mL |
| Clumping | Value = 3 |

Transfection Efficiency

| | |
|---|---|
| % GFP Rep 1 | Greater than or equal to 30% positive cells |
| % GFP Rep 2 | Greater than or equal to 30% positive cells |
| % Viability Rep 1 | Greater than or equal to 60% positive cells |
| % Viability Rep 2 | Greater than or equal to 60% positive cells |

Ambr ®15

| | |
|---|---|
| PDT Ambr ®15 | Greater than or equal to 32 hours |
| Peak VCD Ambr ®15 | Greater than or equal to 3 × 10⁶ cell/mL |
| Day 90% viability Ambr ®15 | Greater than or equal to day 6 |

Although some clones showed cell viability slightly below 90% at P1 after thawing, they were nonetheless selected if able to show higher cell viability on P2. Of the 23 clones, 11 clones were selected to continue to the next characterization step (Step 3) where they were tested for shear stress tolerance in the Ambr®15 system.

### Step 3—Shear Stress

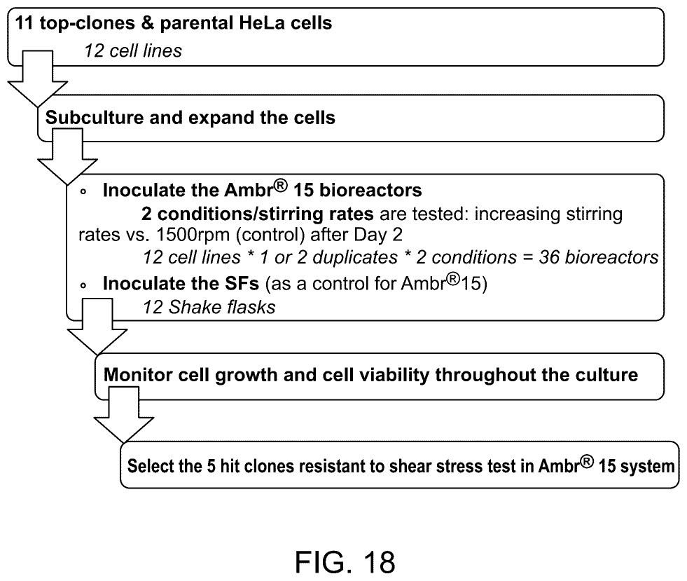

The 11 clones with the best performance identified in Step 2 were thawed and expanded for 2 passages. HeLaS3 parental cells were used as control. The shear stress tolerance test is shown in FIG. 18.

After two passages, clones were seeded in Ambr®15 vessels and cell culture followed the starting conditions:
1. Seeding =0.2×10⁶ cells/ml
2. pH control at 7.2±0.05
3. Constant stirring rate 1500 rpm, downstir
4. Dissolved oxygen (DO)=50%
5. Antifoam: Foam Away diluted 1:10 and added daily

On day 2, the stirring was increased to 1650 rpm, followed by an increase to 1800 and 2000 rpm, on days 3 and 4 respectively (Table 5). On day 6, some clones were still not affected, and as such, the DO setpoint was changed to increase aeration and bubble formation, and consequently the shear rate. Kolmogorov Eddy size calculations are presented in Table 5.

TABLE 5
Stirring rates and Kolmogorov eddy size equivalences
used in shear stress tolerance test.

| Stirring rate (RPM) | Average shear rate (0) | Kolmogorov eddy size |
|---|---|---|
| 1500 | 2497 | 21.9 |
| 1650 | 2881 | 20.4 |
| 1800 | 3283 | 19.0 |
| 2000 | 3845 | 17.6 |
| 2000 and 80% DO | >3845 | <17.6 |

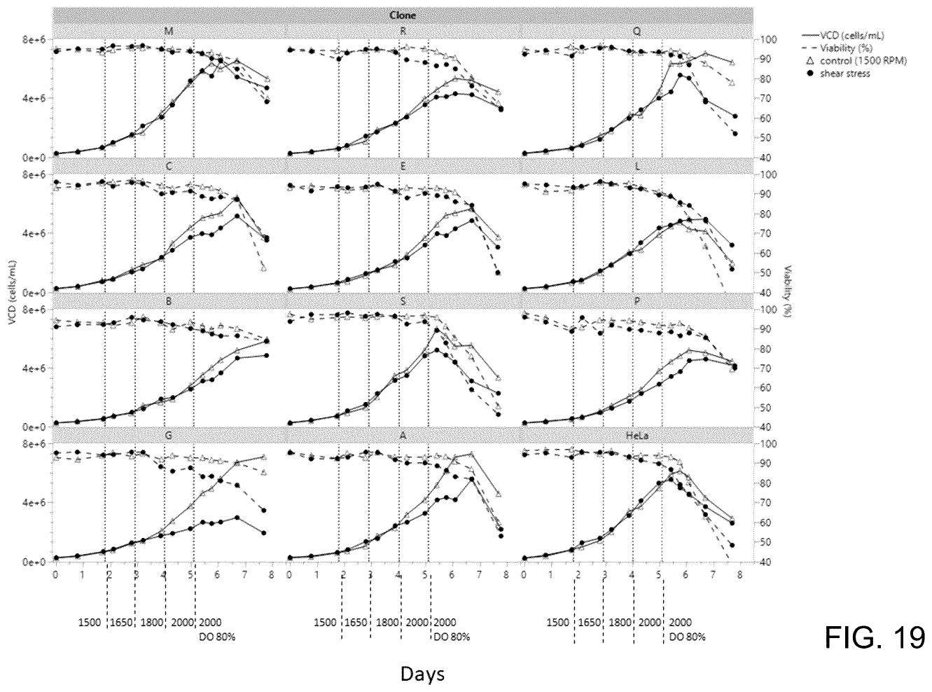

Sampling for cell count and viability determination was performed around 8 h and 24 h after changing the setpoint (stirring or DO). Therefore, the 24 h sampling was right before increasing shear stress again. Until day 2 and after day 6, sampling was performed daily. Selected clones (M, Q, E, S, and P FIG. 19) were considered the 5 top clones.

### Example 6—Characterization of Selected Clones

Following the process described in Example 5, five clones were selected for further characterization. These clones were studied in a shake flask for their PDT and peak VCD. The five clones were also studied in a high throughput, automated cell culture bioreactor system, Ambr® 15, for their PDT and peak VCD and further evaluated for clumping.

Table 6 below shows the comparison of the five clones designated as clone M, R, Q, C and E for their several attributes compared to the HeLa parental cell line.

TABLE 6

| | POT Growth | | | | Clumping scale |
|---|---|---|---|---|---|
| | Curve in | Peak VCD | PDT Growth | Peak VCD | 1-5 subjective |
| | shake flask | Shake Flask | Curve (Ambr ®15) | Arritor 15 | measurement (two |
| | (hours) ± SD | (average, | (hours) ± SD | (average, | operators at |
| Clone | n = 4 | cell/mt) | n = 3 | cell/mL) | two days) |
| HeLa Parental | 24 ± 1 | 6.70E+06 | 27.0 ± 0.4 | 6.30E+06 | 4 |
| Clone M | 24.9 ± 0.5 | 7.50E+06 | 27 ± 1 | 7.50E+06 | 4.8 |
| Clone R | 25.2 ± 0.8 | 7.40E+06 | 28.5 ± 0.5 | 6.20E+06 | 4.2 |
| Clone Q | 24 ± 2 | 9.00E+06 | 26 ± 1 | 7.20E+06 | 3.6 |
| Clone C | 29 ± 1 | 9.00E+06 | 29 ± 1 | 6.20E+06 | 5 |
| Clone E | 29 ± 2 | 9.50E+06 | 28 ± 2 | 6.80E+06 | 5 |

Other attributes of the clones studied were their (i) transfection efficiency and viability, (nucleofector efficiency and viability) and (iii) expression ratio of several immune response gene transcript levels to the HeLaS3 parental levels. The housekeeping gene HRPTI was used as a control.

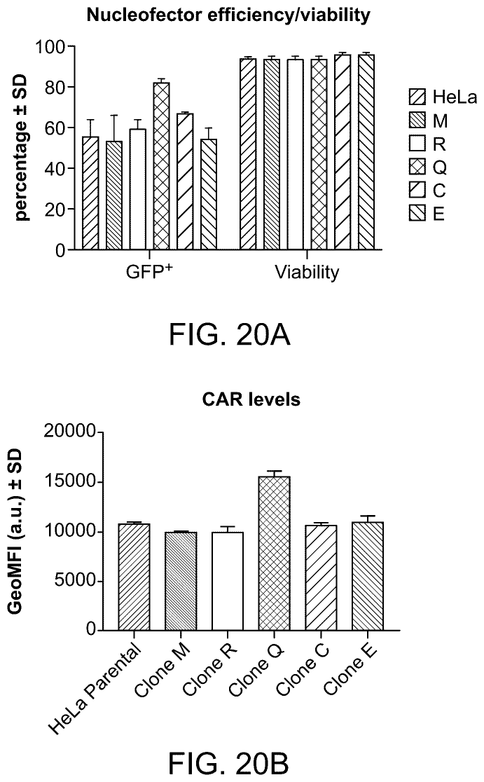

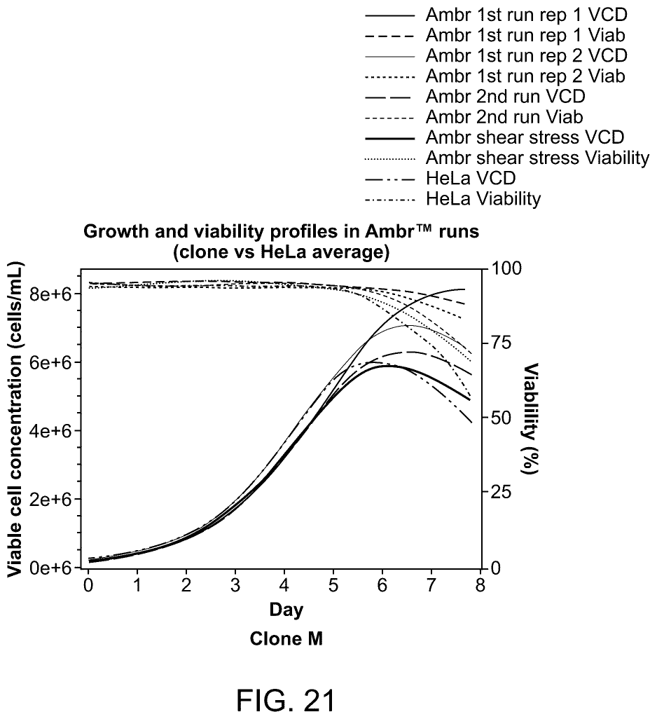

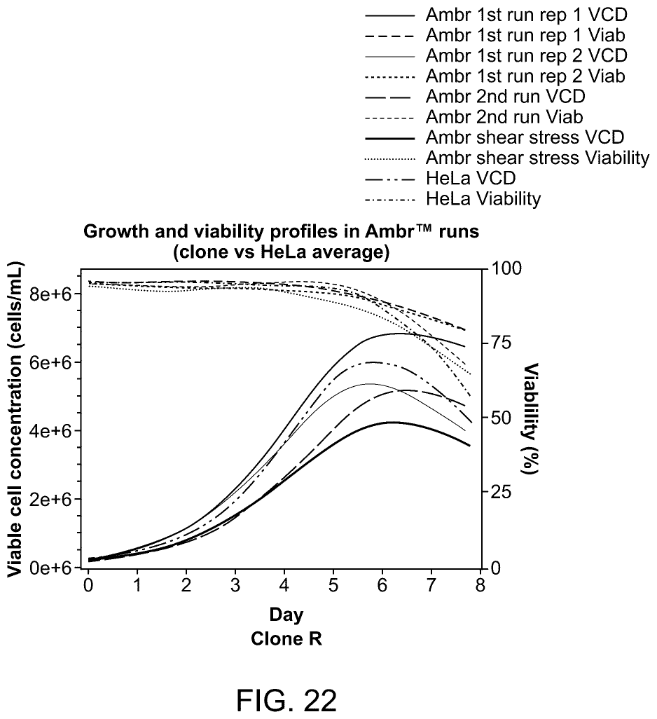

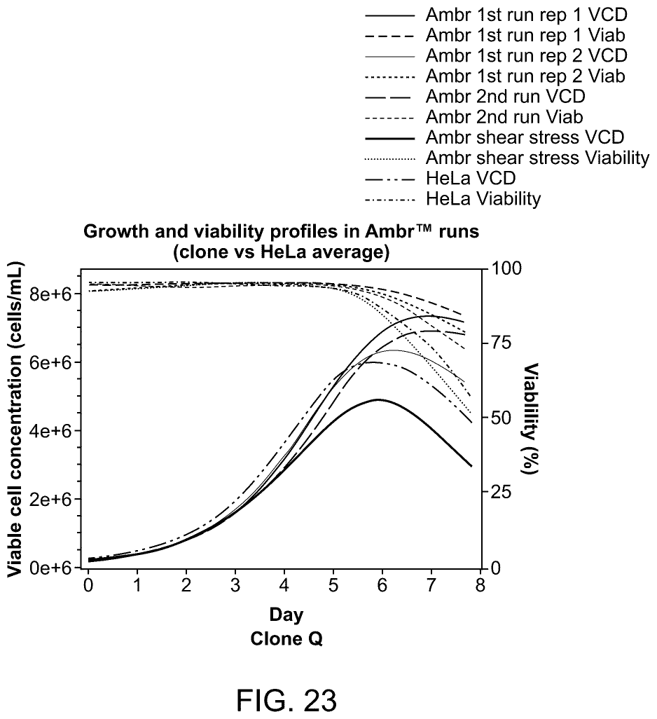

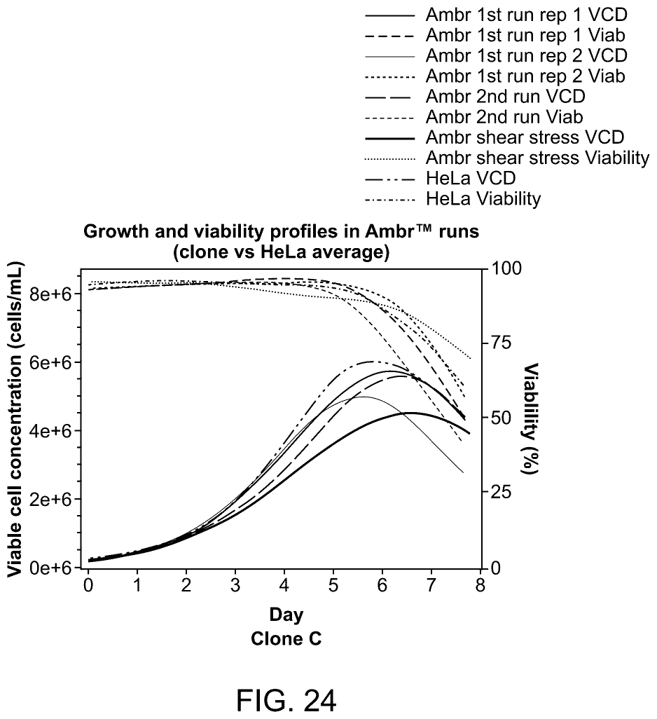

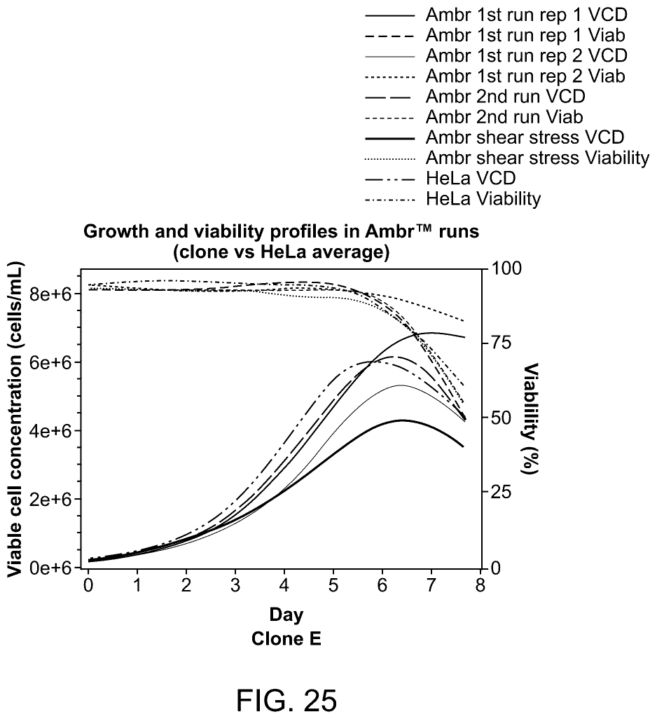

FIG. 20 shows attributes of the top five clones M, R, Q, C, and E related to the nucleofector efficiency and viability (FIG. 20A. Also shown are the growth curves for the various clones: Clone M (FIG. 21); Clone R (FIG. 22); Clone QM (FIG. 23); Clone C (FIG. 24); and Clone M (FIG. 25).

Table 7 shows for selected clones their productivity and robustness as determined by their AAV titer data. The smaller the value recommend for seeding, the more robust was the clone.

TABLE 7

| | Recommended seeding | Titer at MW stage |
|---|---|---|
| Clone | (cells/well) | (vg/ml) |
| Clone M | 1.25K-2.5K | 4.80E+09 |
| Clone B | 1.5K-3K | 7.17E+10 |
| Clone Q | 3K-6-K | 1.32E+10 |
| Clone E | 2K-4K | 2.11E+10 |
| Clone C | 1.24K-2.5K | 1.30E+10 |

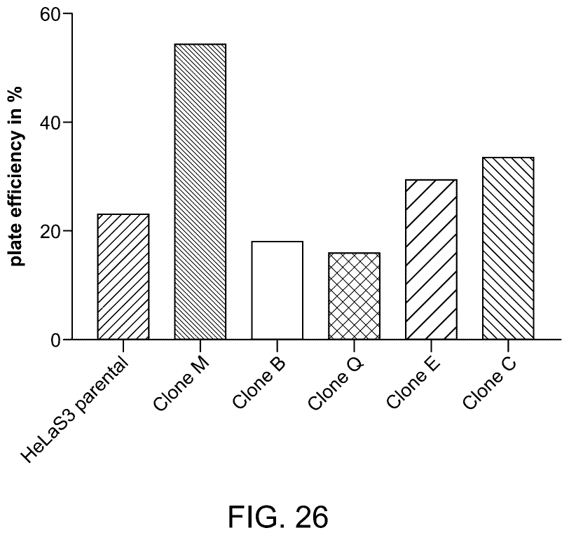

FIG. 26 also shows for selected clones their productivity and robustness at the limiting dilution cloning (LDC) stage as determined by their plate efficiency growth evaluated 2 weeks after seeding. The smaller the valued recommend for seeding, the more robust was the clone.

### Example 7: Generation of Producer Cell Lines

### Thaw and Passaging

The five clones identified in Example 6 were each stored in cryovials in liquid nitrogen tanks (LN2) at 1E7 cells in 1 mL. Vials were transferred from LN2 tank to the lab via a filled cryopod (150° C. and −180° C.). Vials were thawed in a water bath set at 37° C. and kept in the water bath until it was approximately 90% thawed, at which point it was sprayed with ethanol and transferred to a working hood. The vial contents were transferred to either a 15 mL or a 50 mL conical flask, to which 9.2 mLs of fresh EX-CELL® Growth media (EX-CELL® Media and L-Glutamine) were added (final volume upon media addition: 10.2 mLs). Cells were counted on VICELL™ to determine the cell density and viability (200 μl needed for count). Remaining 10 mLs then was spun down and resuspended in a volume of ranging from 10-40 mLs, with a target density of 3E5 cells/mL, in a 125 mL Shake Flask (SF). The SF was kept in a Kuhner shaking incubator at 110 RPM, 80% humidity, 5% CO2, and 37° C. Passaging was done every 3-4 days, where the culture was thoroughly resuspended before cell density and viability counts were done on the VICELL™. Cells were passaged at a seeding density of 2E5 cells/mL for any passage post-initial thaw.

### Transfections

Nucleofections (NTx) are an electroporation-based transfection technique using the Lonza 4D Nucleofector. Nucleofections were carried out 3-days after two passages post thaw. For transfections, population doubling time (PDT) was under 26 hours, and cell viability (CV %) was greater than or equal to 98%. Cells were transfected with defined quantities of the various reagents (DNA/buffer supplements and at a given cell density). Each reaction required 4E6 cells and 6 μg of plasmid DNA (diluted in LONZA nucleofection reagents) following the manufacturer's recommendation. Transfections were done in the appropriate settings for the respective cell type and following an established transfection protocol. Post-NTx, cells were placed either in static incubators (if in a 6 well plate) or a shaking incubator (if in a shake flask). 24 hours and 48 hours post-NTx, the transfection efficiencies for the controls (GFP and Negative, with negative control without any plasmid DNA) were evaluated by a cytometer (CELIGO™), and cell counts for the SFs were calculated on the VICELL™.

### Plating and Selection of Transfected Cells

72 hours post-NTx, counts were done on the VICELL™ for the cell cultures. Cells were seeded in selection medium (EX-CELL® Media, L-glutamine, and puromycin) at 2.5K cells/well in 150 μl in 96 well plates. They were placed in static incubators at 37° C., 80% humidity, and 5% CO2, where they remained until scale up was initiated. Every 7 days, 30 μl of fresh selection medium was added to the wells. Plates were scanned once a week to determine outgrowth patterns, which enables the next step for scale up.

### Scale up

Starting between 14-18 days post-selection, wells were scaled up from 96 well plates to 24 well plates containing 1 mL of fresh selection medium. Scans were evaluated on the day or one day before scale up, and any wells with sufficient colony size (3D outgrowth) were scaled up using an automated liquid handling system. Scale up usually took place over 3 rounds, or until target number of wells scaled up was achieved 1000-1200 total wells).

### Primary Screens

7 days post-scale up, counts were done for the 24 well plates using CELIGO™. Wells with cell density at or higher than 3.5E5 cells were further processed for the primary screen. 100,000 cells were transferred from the 24 well plates to a 96 well V-bottom plate via an automated liquid handling system. After transfer was complete, these plates were spun down at 300 g for 4 min. After the media was spun down, it was aspirated off, and fresh infection medium containing EX-CELL® was added to the wells in a total volume of 200 μl. The entire content of the plate was further transferred to new 96 well plate, followed by incubation at 37° C., 80% humidity, and 10% CO₂ for 72 hours. After 72 hours, the wells were treated with TWEEN® (polysorbate) and benzonase for 1 hour, where they further were incubated under static conditions at 37° C., 80% humidity, and 5% CO₂. Post incubation, 100 μl of the treated sample was transferred to a new 96 well plate, and further processed for viral particle or vector production titers using qPCR (as reported in Martin 2013). Table 8 below shows the titers of selected clones in the primary screen, with viral productivity titers close 1E10.

### Results Scale up and Secondary Screen

Once the qPCR data was available, top producers for each screening round were selected, and were scaled up from 24 wp to T25 culture flasks. As a part of the scale up process, the cells were transferred from T25 flasks to SFs. Cultures exhibiting PDTs at or under 40 hours and CV % at or higher than 90%, were banked and processed for 3 consecutive passaging rounds for secondary screens (5E6 cells in 10 mL of infection medium, shaking conditions). The samples in shake flasks were analyzed for viral productivity by qPCR. Data was reviewed for the secondary screens and overall, the performance was evaluated for each candidate across one primary screen and 3 secondary screens.

Table 8 shows the results of the secondary screens in which the titers improved from the primary screen to the secondary screen. For example, for the Clone M, the highest titer for the primary screen was around 1E10 and the highest average titer observed during the secondary screen was 3E10. Similarly, for the Clone C, the highest titer for the primary screen was around 4E10 and the highest average titer observed during the secondary screen was 1.1E11.

TABLE 8

| | | | Secondary Screen |
|---|---|---|---|
| | | Primary Screen | Results AVG (3 |
| MW | Subclone | Results (vg/mL) | Rounds) (vg/ml) |
| 1139 | B | 3.17E+09 | 1.56E+10 |
| 708 | Q | 3.97E+10 | 5.00E+10 |
| 818 | Q | 2.07E+10 | 6.95E+10 |
| 838 | Q | 1.57E+10 | 2.41E+10 |
| 1227 | Q | 1.38E+10 | 4.25E+10 |
| 140 | G | 1.16E+10 | 4.99E+10 |
| 196 | G | 9.72E+09 | 3.17E+10 |
| 300 | G | 9.47E+09 | 6.07E+10 |
| 340 | G | 6.69E+09 | 1.99E+10 |
| 378 | G | 3.28E+10 | 7.04E+10 |
| 398 | G | 1.18E+10 | 3.56E+10 |
| 409 | G | 6.58E+09 | 2.83E+10 |
| 1035 | G | 7.49E+09 | 2.97E+10 |
| 1539 | G | 2.92E+09 | 1.59E+10 |
| 274 | M | 5.22E+09 | 3.46E+10 |
| 15 | M | 1.04E+10 | 3.28E+10 |
| 157 | M | 5.80E+09 | 3.17E+10 |
| 155 | M | 9.51E+09 | 2.78E+10 |
| 91 | M | 1.02E+10 | 2.22E+10 |
| 1049 | C | 4.20E+10 | 1.14E+11 |
| 298 | C | 1.37E+10 | 8.29E+10 |
| 244 | C | 9.25E+09 | 8.28E+10 |
| 587 | C | 1.16E+10 | 7.64E+10 |
| 307 | C | 7.42E+09 | 4.14E+10 |
| 428 | C | 1.18E+10 | 2.33E+10 |
| 908 | C | 1.57E+10 | 1.54E+10 |
| 645 | C | 1.30E+10 | 3.27E+09 |

Note:
All subclones were seeded at 2.5K Viable Cells/well

1. A host cell derived from a HeLaS3 parental cell line.

2. The host cell of claim 1, wherein the host cell when compared to the parental cell line has about a 0.5% to about a 25% difference in:
a cell doubling time (hours);
a transfection efficiency;
a cell viability; or
a peak viable cell density.

3-4. (canceled)

5. The host cell of claim 1, wherein the host cell has about a 0.5% to about a 10% difference in cell viability after one cell freeze thaw cycle compared to the parental cell line.

6. The host cell of claim 1, wherein when the host cell is transfected with one or more nucleic acid molecules encoding a heterologous transgene flanked by AAV ITRs, an AAV rep, and an AAV cap and has about a 1- to 20-fold difference in Adeno-associated virus (AAV) vector production titer (vg/mL) compared to the parental cell line.

7. A method for selecting a host cell line, comprising:
(a) expanding one or more population(s) of a HeLaS3 parental cell line in a serum-free media;
(b) selecting and isolating one or more single cell clones from step (a);
(c) expanding each of the one or more single cell clones from step (b) in a serum-free media;
(d) selecting from each of the one or more single cell clones from step (c) and analyzing the one or more single cell clones for at least one of the following characteristics:
(i) cell doubling time,
(ii) transfection efficiency,
(iii) peak viable cell density;
(iv) cell outgrowth after seeding at low cell density;
(v) cell viability:
(vi) clumping degree; or
(vii) any combination thereof; and
(e) growing the one or more single cell clones selected from step (d) in a serum-free media.

8. The method of claim 7, wherein step (d) further comprises:
an evaluation of recovery from a cell freeze-thaw cycle;
an evaluation of a degree of cell clumping;
a selection based on a principal component analysis; or
an evaluation the metabolic profile, optionally wherein the evaluation of the metabolic profile comprises measuring depletion of glucose or glutamine or secretion of lactate or any combination thereof into the serum-free media for 1-7 days.

9-12. (canceled)

13. The method of claim 7, wherein the one or more single cell clones from step (d) when compared to the parental cell line has at least one of the following characteristics:
about a 0.5% to about a 25% difference in cell doubling time in hours;
about a 0.5% to about a 25% difference in peak viable cell density;
about a 0.5% to about a 25% difference in percent cell viability after one cell freeze thaw cycle; or
about a 1- to 20-fold difference in AAV vector production titer (vg/mL) when transfected with one or more nucleic acid molecules encoding a heterologous transgene flanked by AAV ITRs, an AAV rep, and an AAV cap and infected with a helper virus.

14-15. (canceled)

16. The method of claim 7, wherein the one or more single cell clones from step (c) has about a 0.5% to about a 25% difference in transfection efficiency compared to the parental cell line.

17. (canceled)

18. The method of claim 7, wherein the host cell line can grow in suspension.

19. A recombinant AAV (rAAV) particle produced by a method comprising:
a) transfecting a host cell line selected using the method of claim 7 under conditions to generate the rAAV particles, wherein the host cell line is transfected with one or more nucleic acid molecules encoding a heterologous transgene flanked by inverted terminal repeats (ITRs), an AAV rep, an AAV cap, and optionally a selectable marker; and expanding the one or more population(s) from the transfected parental cell line;
b) infecting the one or more population(s) with an AAV helper virus or a derivative thereof; and
c) harvesting the rAAV particle.

20. The rAAV particle of claim 19, wherein:
the nucleic acid molecule encoding the AAV cap is derived from AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV V708K, a goat AAV, AAV1/AAV2 chimeric, bovine AAV, or mouse AAV capsid rAAV2/HBoV1 or variants thereof;
the AAV helper virus is adenovirus 5 (Ad5);
the host cell line is infected with a helper virus, optionally wherein the helper virus is an adenovirus that is optionally a human Ad5, a herpes simplex virus, a vaccinia virus, or a cytomegalovirus;
the transfected host cell line is harvested to collect AAV particles; or
the one or more nucleic acid molecules are stably transfected into the host cell line.

21-26. (canceled)

27. A method for generating a producer cell line candidate for production of recombinant AAV (rAAV) particles, the method comprising:
(a) stably transfecting a host cell line that has been selected by the method of claim 7 in a serum free medium with one or more nucleic acids encoding (i) a heterologous transgene flanked by two AAV inverted terminal repeats, (ii) an AAV rep gene and an AAV cap gene to generate a producer cell line; and
(b) infecting the producer cell line with an AAV helper virus to produce rAAV particles, and selecting the producer cell line as a candidate for production of rAAV particles if the producer cell line generates a titer of rAAV particles of at least about 1E9 vg/mL.

28. The method of claim 27, further comprising:
step (c) of expanding the producer cell line of step (b) to a cell density at or higher than 3.5E5 cells; and (d) selecting the producer cell line as a candidate for production of rAAV particles if the producer cell line generates a titer of rAAV particles of at least about 1E9 vg/mL following step (d); and optionally further comprising
step (e) of expanding the cells of step (d) to a cell density at or higher than 3.5E5 cells and selecting the producer cell line as a candidate for production of rAAV particles if the producer cell line generates a titer of rAAV particles of at least about 1E10 vg/mL following step (e).

29. (canceled)

30. The method of claim 27, wherein the rAAV titer is determined by quantitative polymerase chain reaction (qPCR).

31. The method of claim 7, wherein the cell viability or the peak viable cell density is determined after freeze-thawing, shear stress, or a combination thereof,
optionally wherein the cell viability after freeze-thawing or shear stress is greater than or equal to 70% when compared to the parental cell line.

32-33. (canceled)

34. The method of claim 7, wherein:
the method further comprises:
selecting cells that have a cell doubling time less than or equal to 32 hours;
selecting cells that have peak viable cell density greater than or equal to 3×10⁶ cell/mL;
selecting cells that have greater than or equal to 30% transfection efficiency; or selecting cells that have reduced clumping of cells visible to the naked eye
when compared to the parental cell line.

35-37. (canceled)

38. The method of claim 7, wherein:
the serum-free medium is free of any animal derived components;
the serum-free medium comprises medium supplemented with glutamine; or
the serum-free medium is supplemented with about 6 mM of glutamine.

39-42. (canceled)

43. A method of producing a host cell line comprising the steps of:
(a) expanding one or more population(s) of a HeLaS3 parental cell line at a density of 0.5 cells/well in media comprising EX-CELL HeLa growth medium supplemented with 6 mM L-Glutamine, 50% DMEM/F-12 (with 6 mM L-Glutamine), 20% conditioned media and 1× InstiGRO CHO supplement, wherein all components of the media and conditioned media are serum free and free of animal derived components;
(b) selecting and isolating one or more single cell clones from step (a);
(c) expanding each of the one or more single cell clones from step (b) in a serum-free media;
(d) selecting from each of the one or more single cell clones from step (c) for at least one of the following characteristics:
(i) cell viability,
(ii) cell doubling time,
(iii) transfection efficiency,
(iv) peak viable cell density;
(v) clumping degree;
(vi) cell outgrowth after seeding at low cell density; and
(vii) any combination thereof; and
(e) isolating and expanding the one or more single cell clones selected from step (d) in a serum-free media thereby producing the host cell line.

44. A method of generating a producer cell line comprising the steps of
(a) transfecting a host cell line that has been selected by the method of claim 7 with one or more nucleic acids encoding (i) a heterologous transgene flanked by two AAV inverted terminal repeats, (ii) an AAV rep gene and an AAV cap gene, (iii) and an AAV helper gene; and
(b) selecting the producer cell line where the producer cell line generates a titer of rAAV particles of at least about 1E9 to about 1E11 vg/mL.

45. A method for making recombinant adeno-associated virus (rAAV), comprising the steps of:
(a) transfecting a host cell line produced by the method of claim 7 with one or more nucleic acids encoding (i) a heterologous transgene, (ii) an AAV rep gene and an AAV cap gene, and (iii) AAV inverted terminal repeats (ITR);
(b) infecting the host cell with a helper virus; and
(b) isolating the rAAV particles wherein the titer of the rAAV particles produced is at least about 1E9 vg/mL,
optionally wherein the titer of the rAAV particles is from about 1E9 vg/mL to about 1E11 vg/mL.

46. (canceled)
